(19) United States
(12) Patent Application Publication
(10) Pub. No.: US 2024/0269236 A1
(43) Pub. Date: Aug. 15, 2024

(54) METHODS OF TREATING AGE-RELATED FRAILTY WITH INTERLEUKIN-6

(71) Applicant: SONNET BIOTHERAPEUTICS, INC., Princeton, NJ (US)

(72) Inventors: Yves Sagot, Beaumont (FR); Christoph Handschin, Reinach BL (CH); Aurel B. Leuchtmann, Zumikon (CH)

(21) Appl. No.: 18/566,029

(22) PCT Filed: Jun. 3, 2022
(86) PCT No.: PCT/US22/32215

§ 371 Date: Nov. 30, 2023

Related U.S. Application Data
(60) Provisional application No. 63/197,097, filed on Jun. 4, 2021.

(51) Int. Cl. A61K 38/20; A61K 45/06; A61P 21/00

(52) U.S. Cl. CPC A61K 38/204; A61K 45/06; A61P 21/00

(57) ABSTRACT

Provided herein are novel methods for improving muscle performance in a subject with age-related frailty. The methods provided herein generally include a) engaging the subject in an exercise training program; and b) administering to the subject a dose of the recombinant interleukin-6 (IL-6) compositions described herein at regular intervals during the exercise training program. The treatment methods provided herein advantageously improve muscle performance in the subject with age-related frailty. In certain embodiments, the method reduces muscle fatigue during the exercise training program and/or improves muscle endurance.

### CROSS-REFERENCE TO RELATED APPLICATION

The present application claims priority to U.S. Provisional Patent Application No. 63/197,097, entitled “Methods of Treating Age-Related Frailty with Interleukin-6,” filed Jun. 4, 2021, which is hereby incorporated by reference in its entirety for all purposes.

### BACKGROUND

Aging is characterized by a progressive loss of physiological integrity, leading to functional impairments and increased susceptibility to acute and chronic diseases. While regular physical activity is considered to be an anthropological necessity for the maintenance of physiological function throughout life, a sedentary lifestyle is strongly associated with accelerated aging and reduced health span. Even though skeletal muscle is a major driver of pathoetiology, deteriorations of the cardiorespiratory and neuromuscular systems with increasing age also play an important role in modulating the development of severe functional impairments and frailty, as they all contribute to a devastating vicious cycle. Initial decrements in physical performance, fatigue resistance and motor coordination result in unsteady gait and an increased perception of effort during activity, which leads to the avoidance of physical challenges and reduced voluntary exercise. The ensuing muscular disuse further drives performance decline, the loss of muscle mass and strength, favors the development of chronic disorders and increases the risk of falls, disability, and death. Therefore, besides neurodegenerative events, deterioration in functional capacity of skeletal muscles is one of the major causes for loss of independence, admission to nursing homes, morbidity and mortality in the elderly. Of note, due to the high costs associated with care of these patients, sarcopenia and frailty put an escalating burden on health care systems. In light of an expanding global geriatric population, effective interventions to preserve or improve physical performance and functional capacities at advanced ages are thus becoming increasingly important, both on the individual, but also the societal level.

Endurance exercise is an efficient way to improve cardiorespiratory, neuromuscular, and metabolic function, as it requires successful integration of these systems. Endurance exercise training may thus provide a powerful countermeasure for muscular decline, loss of motor coordination and mobility, and metabolic deteriorations with aging. Unfortunately, however, several factors hamper the effectiveness of exercise-based interventions, in particular in the elderly. First, as inactivity and social commitments that require long sedentary periods emerged as common and widely accepted lifestyle choices in modern societies, acceptance of exercise-induced discomfort and pain as well as compliance with long-term training are major obstacles to implement exercise interventions in the human population, young and old. Second, at older age, muscle may show an attenuated immediate and/or an abbreviated response to mechanical loading and thus may require more intense and/or more frequent workouts to significantly adapt. Third, aged muscle may be more vulnerable to exercise-induced injury compared with young muscle and display decreased regenerative capacity, which can limit exercise intensity, the amount of training or the potential for improvement. Fourth, many elderly people have accumulated disuse complications and suffer from morbidity, frailty, and coordinative deficits, which may not allow to manage or tolerate the strenuous or sustained training required to induce significant adaptations. Taken together, there is a need for novel and effective strategies to leverage the beneficial effects of exercise training on physical performance and neuromuscular function in aging. However, effective and safe pharmacological therapies are lacking.

### SUMMARY

Provided herein are novel methods for improving muscle performance in a subject with age-related frailty. The methods provided herein generally include a) engaging the subject in an exercise training program; and b) administering to the subject a dose of the recombinant interleukin-6 (IL-6) compositions described herein at regular intervals during the exercise training program. The treatment methods provided herein advantageously improve muscle performance in the subject with age-related frailty. In certain embodiments, the method reduces muscle fatigue during the exercise training program and/or improves muscle endurance.

In some embodiments, the method reduces muscle fatigue in the subject. In certain embodiments, the method improves endurance. In some embodiments, method improves muscle endurance. In certain embodiments, the method improves aerobic endurance.

In some embodiments, the method improves muscle fatigue resistance. In some embodiments, the method improves muscle recovery in the subject. In certain embodiments, the method improves maximum aerobic capacity in the subject. In some embodiments, the method improves motor coordination. In exemplary embodiments, the method reduces glucose intolerance in the subject. In some embodiments, the method reduces exercise related lactic acidosis in the subject.

In some embodiments, the method increases the expression of a biomarker selected from the following group of biomarkers in the subject: PDK4, LDHA, ATP5F1A, UQCRC2, MTCO1, and NDUFB5. In certain embodiments, the method decreases white adipose tissue, abdominal fat, and/or visceral fat in the subject.

In some embodiments, the subject is at least about 55 years or older.

In certain embodiments, the exercise training program comprises at least one exercise session a week. In some embodiments, each exercise session is at least 15 minutes in length. In certain embodiments, the exercise training program is at least 4 weeks in duration.

In certain embodiments, the subject exerts no more than 65% of the subject's maximum heart rate during each exercise session. In some embodiments, the recombinant IL-6 is administered to the subject prior to each exercise session.

In certain embodiments, the recombinant IL-6 composition is administered to the subject at regular intervals spaced at least 24 hours apart. In some embodiments, the recombinant IL-6 composition is administered at a dose between about 0.05 μg/kg to about 3.0 μg/kg. In some embodiments, the recombinant IL-6 composition is administered at a dose of between about 0.2 μg/kg to about 1.0 μg/kg. In certain embodiments, the recombinant IL-6 composition is administered at a dose between about 3.75 μg to about 225.0 μg. In some embodiments, the recombinant IL-6 composition is administered at a dose of between about 15.0 μg to about 75.0 μg.

In some embodiments, the IL-6 composition further comprises a pharmaceutically acceptable carrier. In certain embodiments, the IL-6 composition is administered subcutaneously, intravenously, intramuscularly or orally. In some embodiments, the IL-6 composition is co-administered with a supplement comprising vitamin D3, Resveratrol Setmelanotide, VIP, or an iron supplement.

### BRIEF DESCRIPTION OF THE DRAWINGS

FIGS. 1A-G are representative graphs that summarize a study showing that long-term recombinant IL-6 treatment is safe and well tolerated. (A) Graphic illustration of the experimental approach of treadmill training and/or rIL-6 injections. (B) Body mass trajectory for all four groups from 0 weeks to 12 weeks (Sed+Saline, n=15; Sed+IL-6, n=16; Ex+Saline, n=13; Ex+IL-6, n=16). (C) Feeding behavior over 48 h (left panel) and average food intake during light and dark phase (right panel) during week 11 of the intervention (Sed+Saline, n=13; Sed+IL-6, n=13; Ex+Saline, n=10; Ex+IL-6, n=15). (D) Habitual activity and (E) core body temperature during light and dark phase at baseline, 6 weeks, and 11 weeks (Sed+Saline, n=7; Sed+IL-6, n=8; Ex+Saline, n=5; Ex+IL-6, n=10). Plasma cytokine levels before (Baseline) and at the end (Terminal) of the study of (E) tumor necrosis factor alpha (TNF-α), (F) interleukin-6 (IL-6) and (G) interleukin-10 (IL-10) (Sed+Saline, n=15; Sed+IL-6, n=15; Ex+Saline, n=13; Ex+IL-6, n=16). (G-I) Y-axis has a logarithmic scale (log 10). Data are mean±SEM (B and C) including individual values (right panel in C and in D and E) and mean and individual values paired with a black line (E-G). Paired Student's t-test (E-G). *P<0.05.

FIG. 2A-F are representative graphs that summarize a study showing that moderate-intensity low-volume endurance training preserves glucose tolerance with aging. Glucose tolerance test (GTT) curves and corresponding area under the curves (AUCs) measured at baseline and after 9 weeks of treatment: (A) Sed+Saline group (n=10), (B) Sed+IL-6 group (n=10), (C) Ex+Saline group (n=8) and (D) Ex+IL-6 group (n=11). Between group comparison of the GTT after 9 weeks: (E) GTT curves of all four groups and (F) corresponding AUCs. Data are mean±SEM (GTT curves), individual values paired with a black line (AUCs of A-D) and box and whiskers depicting the 25th and 75th percentiles (upper and lower perimeters), median (midline), mean (cross), maximum value before upper and lower fence (whiskers) and individual values (values outside upper and lower fence) in (F). Two-way ANOVA (repeated measures) followed by Sidak's multiple comparisons for GTT curves (A-E), paired Student's t-test for AUCs (A-D) and ordinary one-way ANOVA followed by Dunnett's multiple comparisons (F). *P<0.05, **P<0.01. In E: *P<0.05 Sed+Saline vs. Ex+IL-6; #P<0.05 Sed+Saline vs. Ex+Saline.

FIGS. 3A-E are representative graphs that summarize a study showing that moderate-intensity low-volume endurance training preserves \dot{V}O₂ₚₑₐₖ during aging. (A) Graphical representation of the protocol used to assess peak oxygen uptake (\dot{V}O₂ₚₑₐₖ). (B) Blood lactate levels before (Basal) and after (Exhausted) the ramp-sprint test at baseline and after 10 weeks (retest). (C) \dot{V}O₂ₚₑₐₖ values at baseline and after 10 weeks. (D) Maximum speed reached and (E) distance covered until exhaustion during the retest after 10 weeks into the intervention. (B-D) Sed+Saline, n=6; Sed+IL-6, n=7; Ex+Saline, n=6; Ex+IL-6, n=5. Data are mean±SEM (B), individual paired values connected with a black line (C), or box and whiskers depicting the 25th and 75th percentiles (upper and lower perimeters), median (midline), mean (cross), maximum value before upper and lower fence (whiskers) and individual values (values outside upper and lower fence) in (D and E). One-way ANOVA followed by multiple comparisons. *P<0.05, **P<0.01, ***P<0.001 baseline vs. 10 weeks within group; #P<0.05, ##P<0.01, Sed+Saline vs. Ex+Saline or Ex+IL-6 at 10 weeks; δδP<0.01 Sed+IL-6 vs. Ex+Saline or Ex+IL-6 at 10 weeks.

FIGS. 4A-J are representative graphs that summarize a study showing that IL-6 combined with moderate-intensity low-volume endurance training improves treadmill running capacity and muscle fatigue resistance in situ. (A) Graphical representation of the long duration incremental step protocol used to evaluate running capacity. (B) Distance covered until exhaustion at baseline and after 12 weeks. (C) Dropout plot of the retest after 12 weeks showing the percentage of mice running at indicated distances. (D) Time to exhaustion, (E) peak power (Ppeak) achieved and (F) total work (Wtot) performed at baseline and after 12 weeks. (G) Blood lactate levels in basal and exhausted state and (H) the difference exhausted-basal (A) at baseline and after 12 weeks of treatment. (B-G) Sed+Saline, n=15; Sed+IL-6, n=16; Ex+Saline, n=13; Ex+IL-6, n=15. (I) Average curves showing the force decline during a 4 min in situ muscle fatigue protocol on the m. tibials anterior and of muscle force recovery up to 3 min after fatigue with (J) area under the curves corresponding to the force decline during muscle fatigue (Sed+Saline, n=14; Sed+IL-6, n=15; Ex+Saline, n=12; Ex+IL-6, n=14). Data are presented as mean (bars) and paired individual values connected with a black line (B and D-E), mean±SEM (G-I), and box and whiskers depicting the 25th and 75th percentiles (upper and lower perimeters), median (midline), mean (cross), maximum value before upper and lower fence (whiskers) and individual values (values outside upper and lower fence) in (I). Paired Student's t-test (B and D-F), Logrank test for trend (C), two-way ANOVA (H), and one-way ANOVA (I) followed by Sidak's multiple comparisons. *P<0.05, **P<0.01, ***P<0.001. In G: *P<0.05, **P<0.01, ***P<0.001 basal vs. exhausted within group; #P<0.05, ##P<0.01, ###P<0.001 Baseline exhausted vs. 12 weeks exhausted within group; 8P<0.05, 888P<0.001 Sed+Saline 12 weeks exhausted vs. Ex+Saline or Ex+IL-6 12 weeks exhausted. In (H): *P<0.05, **P<0.01, ***P<0.001 Ex+IL-6 vs. Sed+Saline.

FIGS. 5A-J are representative graphs that summarize a study showing that IL-6 treatment improves gait and motor coordination in endurance-trained mice. (A-I) Parameters of quantitative gait analysis at baseline and after 12 weeks of the intervention: (A) average body speed (cm/s), (B) cadence (number of steps/s), (C) average stride length (in cm) of hind and fore limbs. (D-I) Swing speed (cm/s), duration (s) of swing phase and duration (s) of stand time of fore limbs (D-F) and hind limbs (G-I). (A-I) Sed+Saline, n=9; Sed+IL-6, n=9; Ex+Saline, n=7; Ex+IL-6, n=11. (J) Motor coordination assessed by challenging mice with an accelerated Rotarod test at baseline and after 11 weeks of the intervention (Sed+Saline, n=8; Sed+IL-6, n=9; Ex+Saline, n=7; Ex+IL-6, n=11). Data are presented as individual points and paired values connected with a black line. Paired Student's t-test. *P<0.05, **P<0.01, ***P<0.001

FIGS. 6A-F are representative graphs that summarize a study showing that IL-6 treated endurance-trained mice exhibit increased expression of PDK4. Relative m. quadriceps femoris mRNA levels of (A) mitochondrial genes of the electron transport chain [complex (C) I-V, Cyc] and tricarboxylic acid (TCA) cycle and (B) of pyruvate dehydrogenase kinase 4 (Pdk4), pyruvate dehydrogenase E1 component subunit alpha (Pdha1) and pyruvate dehydrogenase phosphatase 1 (Pdp1). Sed+Saline, n=9; Sed+IL-6, n=9; Ex+Saline, n=7; Ex+IL-6, n=11. Expression values were determined by qPCR and normalized to Tbp. Data are shown as the mean fold-change #SEM relative to the expression of the control (Sed+Saline) set to 1. Relative m. quadriceps femoris OXPHOS (C), NDUFB5 (D) and PDK4 (E) protein levels assessed by western blot (n=6 per group). Target band intensities were normalized to the loading control (eEF2, a-Actinin or Ponceau stain) and data are shown as the mean fold-change±SEM relative to the control (Sed+Saline) set to 1. One-way ANOVA followed by Sidak's multiple comparisons (A-E). *P<0.05, **P<0.01, ***P<0.001.

FIGS. 7A-D are representative graphs, showing equal metabolic parameters under non-exercise conditions. (A) oxygen uptake (VO₂), (B) carbon dioxide uptake (VCO₂), (C) respiratory exchange ratio (RER; VCO₂/VO₂) and (D) heat production over 48 h (left panels) and averaged for light and dark phase (right panels) during week 11 of the intervention (Sed+Saline, n=13; Sed+IL-6, n=13; Ex+Saline, n=10; Ex+IL-6, n=15). Data are mean±SEM (left panels A-D) including individual values (right panels in A-D).

FIGS. 8A-K are representative graphs, showing normal blood biochemistry in IL-6 treated mice. Blood plasma levels of (A) alanine aminotransferase (ALAT) (B) aspartate aminotransferase (ASAT), (C) albumin, (D) lactate dehydrogenase (LDH), (E) total cholesterol, (F) low-density lipoprotein (LDL), (G) high-density lipoprotein (HDL), (H) triglycerides and (I) lipase for all four groups after 12 weeks of treatment. Plasma levels of (J) interferon-gamma (INF-y) and (K) interleukin-beta (IL-B) before (Baseline) and at the end (Terminal) of the study (Sed+Saline, n=15; Sed+IL-6, n=15; Ex+Saline, n=13; Ex+IL-6, n=16). (J and K) Y-axis has a logarithmic scale (log 10). Data are presented as box and whiskers depicting the 25th and 75th percentiles (upper and lower perimeters), median (midline), mean (cross), maximum value before upper and lower fence (whiskers) and individual values (values outside the upper or lower fence) for (A-I) or individual values paired with a black line (J and K). One-way ANOVA followed by Tukey's multiple comparisons (A-I) and paired Student's t-test (J and K). *P<0.05.

FIGS. 9A-G are representative graphs, showing the effects of moderate-intensity low-volume endurance training and IL-6 treatment on body composition. (A) Absolute fat mass at baseline and after 12 weeks. (B) Relative fat mass change from baseline to 12 weeks in percent. (C) Absolute lean mass at baseline and after 12 weeks. (D) Relative lean mass change from baseline to 12 weeks in percent. (E) Percent body fat at baseline and after 12 weeks. (A-E) Sed+Saline, n=6; Sed+IL-6, n=7; Ex+Saline, n=6; Ex+IL-6, n=6. (F) Fat depot mass upon dissection of epididymal white adipose tissue (eWAT) and anterior subcutaneous white adipose tissue (sWAT). (G) Individual muscle mass of m. quadriceps femoris (QUAD), m. gastrocnemius (GAS), m. triceps brachii (TRI), m. extensor digitorum longus (EDL), m. soleus (SOL) and m. plantaris (PLAN) upon dissection (Sed+Saline, n=15; Sed+IL-6, n=16; Ex+Saline, n=13; Ex+IL-6, n=16). Data are mean including individual paired values connected with a black line (A-E) or individual values and mean±SEM (F and G). Paired Student's t-test (A-C) and one-way ANOVA followed by Tukey's multiple comparisons (individual tissues in F). *P<0.05, **P<0.01, ***P<0.001 as indicated.

FIGS. 10A-I provides a summary of experiments depicting, that increase in muscle performances is accompanied with an increase of muscle LDH-H (10A-B) and fatigue recovery (10C) but not on contractile properties in situ and grip strength in vivo (10D-I) . . . (A) Representative western blot of m. quadriceps femoris lactate dehydrogenase heart subunit (LDH-H) and corresponding band quantification. LDH-H values are normalized to α-Actinin and expressed relative to Sed+Saline (n=6 per group). (B) Area under the curves (AUCs) of the recovery period following the fatigue protocol. Electrical sciatic nerve stimulation evoked (C) absolute muscle force-frequency relationship, (D) specific twitch force and (E) specific tetanic force. (F) Masses and lengths of the m. tibialis anterior used to calculate specific forces. (G) Normalized in vivo muscle force estimated by measuring peak force of whole limb grip (kgf, kilogram-force; one kgf is equal to 9.806650 N). (H) Single twitch time-to-peak tension and (I) single twitch half-relaxation time. Sed+Saline, n=14; Sed+IL-6, n=15; Ex+Saline, n=12; Ex+IL-6, n=14. Data are presented as mean±SEM (A and C) including individual values (F), box and whiskers depicting the 25th and 75th percentiles (upper and lower perimeters), median (midline), mean (cross), maximum value before upper and lower fence (whiskers) and individual values (values outside the upper or lower fence) in (B, D, E and G-I). One-way ANOVA followed by Sidak's multiple comparisons (A). *P<0.05, **P<0.01.

FIGS. 11A-C are representative graphs showing additional parameters obtained with the CatWalk voluntary gait analysis system described herein. (A-C) Additional parameters of quantitative voluntary gait analysis at baseline and after 12 weeks of the intervention: (A) Average duration (s) of a step cycle and base of support i.e., distance (cm) between paws of (B) fore limbs and (C) hind limbs (Sed+Saline, n=9; Sed+IL-6, n=9; Ex+Saline, n=7; Ex+IL-6, n=11). Data are presented as individual points and paired values connected with a black line. Paired Student's t-test. *P<0.05.

FIGS. 12A-C are representative graphs showing the expression of fatty acid transport and synthesis and glucose metabolism related genes. Relative m. quadriceps femoris mRNA levels of (A) fatty acid transport and synthesis and (B) glucose metabolism related genes (Sed+Saline, n=9; Sed+IL-6, n=9; Ex+Saline, n=7; Ex+IL-6, n=11). Expression values were determined by qPCR and normalized to Tbp. Data are shown as the mean fold-change±SEM relative to the expression of the control (Sed+Saline) set to 1. (C) Relative m. quadriceps femoris STAT3 protein levels assed by western blot (n=6 per group). Target band intensities were normalized to the loading control (Ponceau S stain) and data are shown as the mean fold-change±SEM relative to the control (Sed+Saline) set to 1. One-way ANOVA followed by Sidak's multiple comparisons (C). *P<0.05, **P<0.01, ***P<0.001.

### DETAILED DESCRIPTION

### I. Overview

Provided herein are novel methods for improving muscle performance in a subject with age-related frailty in a subject. In some embodiments, the method includes: a) engaging the subject in an exercise training program; and b) administering to the subject a dose of the recombinant interleukin-6 (IL-6) compositions described herein at regular intervals during the exercise training program.

Previous studies have described a correlation between elevated plasma IL-6 and frailty severity. See, e.g., Ma et al., Clin Interv Aging 13:2013-2020 (2018); Johansen et al., Clin J Am Soc Nephrol 12(7):1100-1108 (2017); Santos Morais Junior et al., J Aging Res. 2020:6831791 (2020); and Marcos-Perez et al., Geroscience 42(6):1451-1473 (2020). In addition, previous studies have shown that exercise improves frailty parameters and decrease IL-6 levels. See, e.g., Bautumans et al., Exp Gerontol 146:111236 (2021); Sadjapong et al., Int J Environ Res Public Health 17(11):3760 (2020); Grosicki et al., J Frailty Aging 9(1):57-63 (2020); Hangelbroek et al., Exp Gerontol. 106:154-158 (2018); and Córdova et al., Neuroimmunomodulation. 18(3): 165-70 (2011). As disclosed herein, however, IL-6 administered at regular intervals during exercise surprisingly reduces muscle fatigue associated with age frailty and improves muscle endurance (e.g., muscle endurance and/or aerobic endurance) and/or muscle recovery after exercise.

The methods described herein are useful for the treatment of any subject that exhibits muscle fatigue associated with age-related frailty. In some embodiments, the subject is at least 30 years old, 35 years old, 40 years old, 45 years old, 50 years old, 55 years old, 60 years old, 65 years old, 70 years old, 75 years old, 80 years old, 85 years old, 90 years old, 95 years old, or 100 years old. In certain embodiments, the subject is between 40-50 years old, 50-60 years old, 60-70 years old, 70-80 years old, 80-90 years old, or 90-100 years old. In certain embodiments, the subject is between 55-90 years old.

Aspects of the subject methods are described in detail below.

### II. Interleukin-6 Composition

In the subject methods provided herein, an IL-6 treatment is administered at regular intervals to the patient in conjunction with engagement with an exercise training program. Such methods advantageously help to treat muscle fatigue linked to age-related frailty in the patient.

In some embodiments, the IL-6 treatment includes a recombinant IL-6, or a biologically active IL-6 variant or biologically active fragment thereof. In particular embodiments, the IL-6 is a human IL-6, or a biologically active human IL-6 variant or biologically active fragment thereof.

As used herein, “IL-6,” “IL6,” “BSF2,” “HGF,” “HSF,” “IFNB2,” “BSF-2,” “B cell stimulatory factor 2,” “CDF,” “IFN-beta-2,” and “interleukin 6” (Genbank Accession numbers: NM_000600, NM_001318095, NP_000591 and NP_001305024 (human IL-6); and NM_031168, NM_001314054, NP_001300983, and NP_112445 (mouse IL-6)) all refer to an interleukin that acts as both a pro-inflammatory cytokine and an anti-inflammatory myokine. IL-6 signals though a cell-surface type I cytokine receptor complex that consists of the IL-6Rα chain (also referred to as “gp 80”) and gp 130 (also referred to as “CD130”).

Interleukin-6 (IL-6) is a multifunctional cytokine produced and secreted by several different cell types. IL-6 is a 20 to 26 kDa glycoprotein having 185 amino acids that has been cloned previously (May et al, (1986); Zilberstein et al, (1986); Hirano et al, (1986)). IL-6 is secreted by a number of different tissues including the liver, spleen, and bone marrow and by a variety of cell types including monocytes, fibroblasts, endothelial, B- and T-cells. IL-6 is activated at the transcriptional level by a variety of signals including viruses, double stranded RNA, bacteria and bacterial lipopolysaccharides, and inflammatory cytokines such as IL-1 and TNF.

The biological activities of IL-6 are mediated by a membrane receptor system comprising two different proteins: IL-6 receptor (gp80) and gp130. Soluble forms of IL-6R gp80 (sIL-6R), corresponding to the extracellular domain of gp80, are natural products of the human body found as glycoproteins in blood and in urine (Novick et al, 1990, 1992). An exceptional property of sIL-6R molecules is that they act as potent agonists of IL-6 on many cell types including human cells. Even without the intracytoplasmic domain of gp80, sIL-6R is still capable of triggering the dimerization of gp130 in response to IL-6, which in turn mediates the subsequent IL-6-specific signal transduction and biological effects (Murakami et al, 1993). sIL-6R has two types of interaction with gp130 both of which are essential for the IL-6 specific biological activities (Halimi et al., 1995), and the active IL-6 receptor complex was proposed to be a hexameric structure formed by two gp130 chains, two IL-6R and two IL-6 ligands (Ward et al., 1994; Paonessa et al, 1995).

In some embodiments, the IL-6 used in the subject methods is a biologically active variant of IL-6. Biologically active variant IL-6s include one or more of the amino acid residues of the naturally occurring components of IL-6 are replaced by different amino acid residues, or are deleted, or one or more amino acid residues are added to the original sequence of an IL-6, without changing considerably the activity of the resulting products as compared with the original IL-6. These variant IL-6s are prepared by known synthesis and/or by site-directed mutagenesis techniques, or any other known technique suitable therefore.

In some embodiments, the IL-6 used in the subject methods exhibit substantially similar, or even better, activity to wild-type IL-6 (e.g., human IL-6). In some embodiments, biological activity of IL-6 is determined as the capability of binding to the gp80 portion of the IL-6 receptor and/or the capability of inducing hepatocyte proliferation, it can be considered to have substantially similar activity to IL-6. Thus, it can be determined whether any given IL-6 variant has at least substantially the same activity as IL-6 by means of routine experimentation comprising subjecting hepatocytes to such mutein, and to determine whether or not it induces hepatocyte proliferation e.g. by measuring BrdU or labelled methionine uptake or just by counting non treated control cells and cells treated with wild-type IL-6. An Enzyme Linked ImmunoSorbent Assay (ELISA) type assay for measuring the binding of IL-6R/IL-6 chimera to gp130 has been described in detail, for example, WO 99/02552. In particular embodiments, an IL-6 is considered to have substantially similar biologically activity to IL-6 if it has substantial binding activity to its binding region of gp80.

In a preferred embodiment, the variant IL-6 at least 40% identity or homology with the sequence of mature wild-type human IL-6. In some embodiments, the variant IL-6 has at least 50%, at least 60%, at least 70%, at least 80%, or at least 90% identity or homology thereto. In some embodiments, the IL-6 used in the subject methods includes at least 95%, 96%, 97%, 98%, 99% or 100% identity to wild-type human IL-6.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus, for instance, programs available in the Wisconsin Sequence Analysis Package, version 9.1 (Devereux J et al. 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990; Pearson 1988). Sequence identity between two similar sequences can also be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc) are used. In one embodiment, sequence identity is done using the BLAST algorithm, using default parameters.

In some embodiments, the IL-6 used in the subject methods described herein includes one or more “conservative” substitutions. Conservative amino acid substitutions of IL-6 may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Examples of production of amino acid substitutions in proteins which can be used for obtaining variants of IL-6 polypeptides, for use herein include any known method steps, such as presented in U.S. Pat. Nos. 4,588,585; 4,737,462; 5,116,943; 4,965,195; 4,879,111; 5,017,691; and 4,904,584. Specific IL-6 variants, which are useful herein have been described, for example, in WO1994003492, U.S. Pat. Nos. 5,681,723, 5,789,552, which is incorporated by reference in relevant parts relating to IL-6 variants.

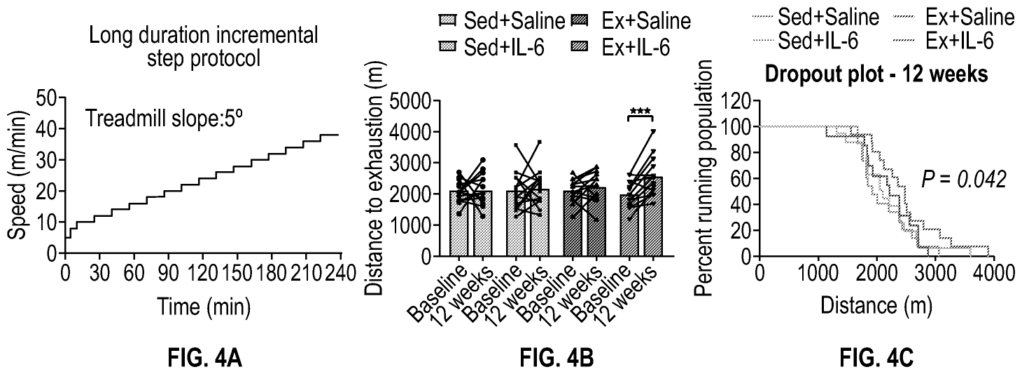

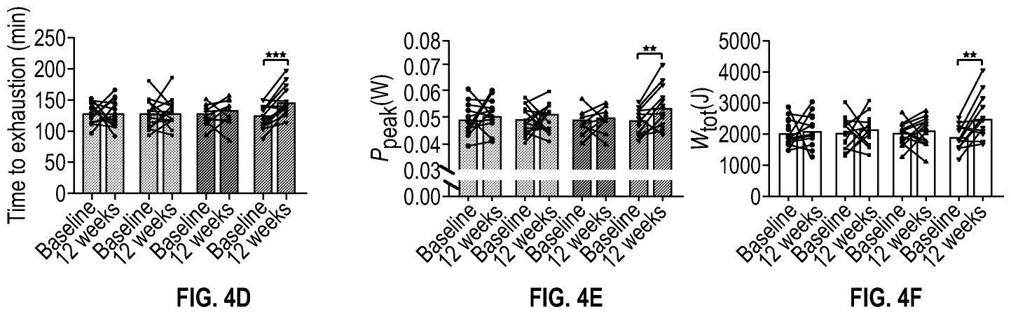

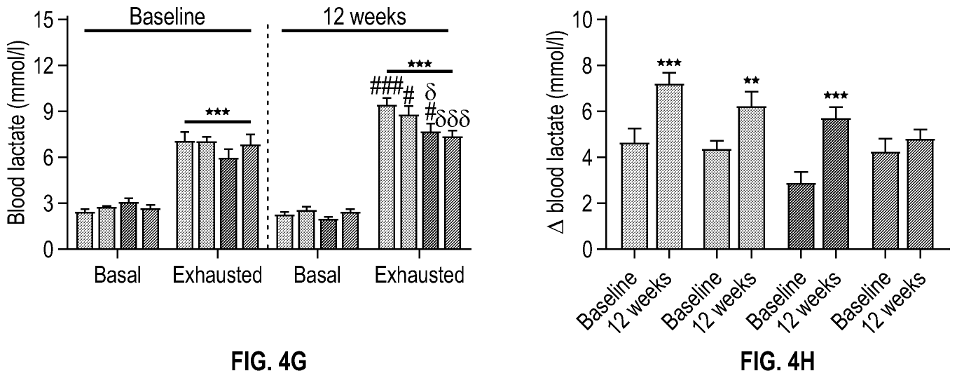

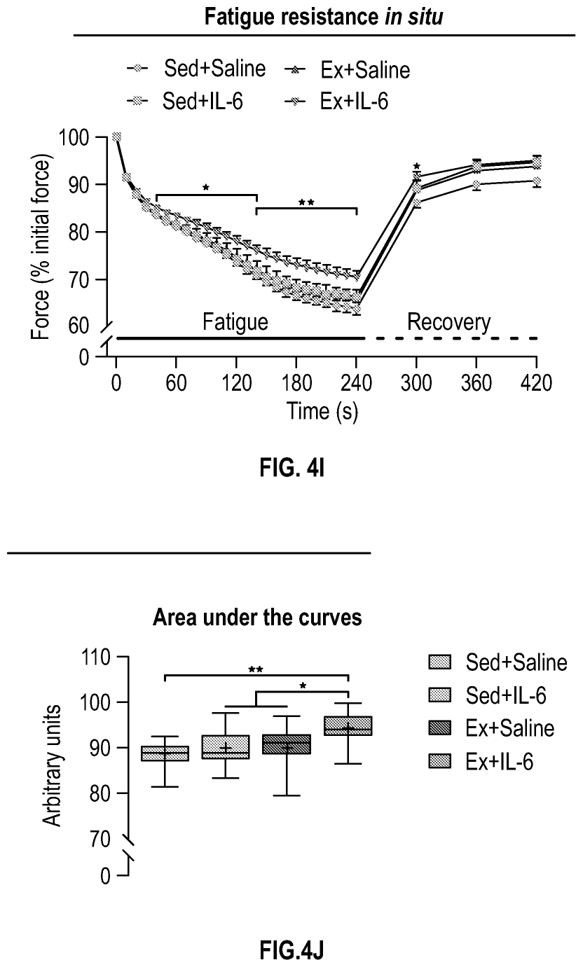

In some embodiments, the IL-6 used in the subject methods is fused to a soluble IL-6 receptor. Chimeric IL-6R/IL-6 molecules linking the soluble IL-6 receptor and IL-6 together have been developed (Chebath et al. Eur Cytokine Netw. 8(4):359-65 (1997)). The chimeric IL-6R/IL-6 molecules were generated by fusing the entire coding regions of the cDNAs encoding the soluble IL-6 receptor (sIL-6R) and IL-6 (see FIG. 4). Recombinant IL-6R/IL-6 was produced in CHO cells (Chebath et al, Eur Cytokine Netw. 1997, WO99/02552). The IL-6R/IL-6 binds with a higher efficiency to the gp 130 chain in vitro than does the mixture of IL-6 with sIL-6R (Kollet et al, Blood. 1999 Aug. 1; 94(3):923-31).

The IL-6 and IL-6R/IL-6 described herein may be produced in any adequate eukaryotic or prokaryotic cell type, like yeast cells, insect cells, bacteria, and the like. In one embodiment, IL-6 is produced in mammalian cells, such as in genetically engineered CHO cells as described in WO 99/02552.

In some embodiments, the IL-6 used in the subject methods is not glycosylated. Advantageously, the molecule can then be produced in bacterial cells, which are not capable of synthesizing glycosyl residues, but usually have a high yield of produced recombinant protein. The production of non-glycosylated IL-6 has been described in detail in EP504751B1, which is incorporated by reference in pertinent parts for teaching methods for making non-glycosylated IL-6.

### III. Compositions for In Vivo Administration

Formulations of the IL-6 used in accordance with the methods provided herein are prepared for storage by mixing an IL-6 having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA or DPTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, polyethylene glycol (PEG) and/or polysorbate.

The formulation herein may also contain more than one active compound as necessary for the particular indication being treated, preferably those with complementary activities that do not adversely affect each other.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the IL-6 composition provided herein, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated the subject IL-6 compositions provided herein remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

IV. Administrative modalities

The subject IL-6 compositions provided herein are administered to a subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, intrarectal, or inhalation routes. Intravenous or subcutaneous administration of the IL-6 composition is preferred.

### V. Exercise Training Program

The IL-6 compositions provided herein are administered in conjunction with an exercise training program. In some embodiments, the exercise training program includes one or more endurance training exercises. Exemplary endurance training exercise include, but are not limited to, walking, running, jogging, dancing, swimming, biking, climbing, and jumping rope.

In some embodiments, the exercise training program includes resistance training exercises. In certain embodiments, the resistance training exercises include weights. Exemplary resistance training exercises with weights include, but are not limited to, bicep curls, shoulder press, bench press, barbell squat, and bent over row. In some embodiments, the resistance training exercise is performed without weights. Exemplary resistance exercises that are performed without weights include but are not limited to, planks, body weight squats, walking lunges, pushups, chin-ups, pull-ups, and sit-ups. In some embodiments, the exercise training program includes concentric and/or eccentric exercises.

In some embodiments, the exercise training program includes a combination of endurance and resistance training exercises. In some embodiments, the training exercise training program includes 1, 2, 3, 4, 5, 6, 7, 8, 9, 10 or more different exercises.

In some embodiments, the patient engages in exercise that is at least 30%, 35%, 40%, 45%, 50%, 55%, 60%, 65%, 70%, 75%, 80%, 85% or 90% the patient's maximum heart rate. In some embodiments, the patient engages in exercise that is between 30%-50%, 40%-60%, 50%-70%, and 60%-80%, 30%-60%, 40%-70%, 50%-80% maximum heart rate. In some embodiments, the patient engages in exercise that is no more than 50%, 55%, 60%, 65%, 70%, 75%, 80%, 85% or 90% the patient's maximum heart rate.

In some embodiments, the exercise training program comprises exercise sessions wherein one or more endurance training exercises are performed. In certain embodiments, each exercise session is at least 10 minutes, 15 minutes, 20 minutes, 25 minutes, 30 minutes, 45 minutes, 60 minutes, 90 minutes, or 120 minutes long. In certain embodiments, each exercise session is no more than 15 minutes, 20 minutes, 25 minutes, 30 minutes, 45 minutes, 60 minutes, 90 minutes, or 120 minutes long. In some embodiments, exercise sessions are performed at least once a week, twice a week, three times a week, four times a week, five times a week, six times a week or seven times a week. In certain embodiments, the exercises are performed for at least one week, two weeks, three weeks or four week. In certain embodiments, the exercises are performed for at least 1, 2, 3, 4, 5, 6, 7, 8, 9, 10, 11, or 12 months. In certain embodiments, the exercises are performed at least 1, 2, 3, 4, 5, 6, 7, 8, 9 or 10 years.

During each exercise session, measurements are taken to assess for improvements in endurance during the course of the treatment program. The measurements taken and metrics assessed will depend on the exercises performed during the exercise sessions. For example, in embodiments wherein the exercise training program includes endurance training, the patient can be assessed for the length of time the patient engaged in the endurance training for a particular session at a particular pace without rest. In some embodiments, wherein the exercise is resistance training, the number of repetitions without breaking from and the amount of resistance used is assessed. The measurements are recorded through the treatment program and improvements in performance of the particular exercise performed are monitored through the course of the treatment program.

### VI. Biomarkers

In addition to a reduction in muscle fatigue and an increase in endurance, patients treated with the subject methods can exhibit an increase in one or more biomarkers. Biomarkers that can be increased by the methods provided herein include, but are not limited to, PDK4 (Pyruvate Dehydrogenase Kinase Isoform 4), LDHA (L-lactate dehydrogenase A chain), ATP5F1A (ATP synthase subunit alpha, mitochondrial), UQCRC2 (Cytochrome b-c1 complex subunit 2, mitochondrial), MTCO1 (Cytochrome c oxidase subunit 1), and/or NUDFB5 (NADH dehydrogenase [ubiquinone] 1 beta subcomplex subunit 5, mitochondrial) or combinations thereof.

In some embodiments, a patient treated using the subject methods exhibits an increase in PDK4 expression. PDK4 is a member of the PDK/BCKDK protein kinase family and encodes a mitochondrial protein with a histidine kinase domain. This protein is located in the matrix of the mitochondria and inhibits the pyruvate dehydrogenase complex by phosphorylating one of its subunits, thereby contributing to the regulation of glucose metabolism. Expression of this gene is regulated by glucocorticoids, retinoic acid and insulin. PDK4 plays a key role in regulation of glucose and fatty acid metabolism and homeostasis via phosphorylation of the pyruvate dehydrogenase subunits PDHA1 and PDHA2. Phosphorylation of the subunits inhibits pyruvate dehydrogenase activity, and thereby regulates metabolite flux through the tricarboxylic acid cycle, down-regulates aerobic respiration and inhibits the formation of acetyl-coenzyme A from pyruvate. Inhibition of pyruvate dehydrogenase decreases glucose utilization and increases fat metabolism in response to prolonged fasting and starvation. PDK4 functions in maintaining normal blood glucose levels under starvation, and is involved in the insulin signaling cascade. Further, via its regulation of pyruvate dehydrogenase activity, PDK4 functions in maintaining normal blood pH and in preventing the accumulation of ketone bodies under starvation. In the fed state, PDK4 mediates cellular responses to glucose levels and to a high-fat diet. PDK4 regulates both fatty acid oxidation and de novo fatty acid biosynthesis. PDK4 further functions in the generation of reactive oxygen species, protects detached epithelial cells against anoikis, plays a role in cell proliferation via its role in regulating carbohydrate and fatty acid metabolism.

In some embodiments, a patient treated using the subject methods exhibits an increase in LDHA (L-lactate dehydrogenase A chain) expression. Lactate dehydrogenase A catalyzes the inter-conversion of pyruvate and L-lactate with concomitant inter-conversion of NADH and NAD+. LDHA is found in most somatic tissues, though predominantly in muscle tissue and tumors, and belongs to the lactate dehydrogenase family. It has long been known that many human cancers have higher LDHA levels compared to normal tissues. It has also been shown that LDHA plays an important role in the development, invasion and metastasis of malignancies.

In some embodiments, a patient engaged in the subject methods exhibits an increase in ATP5F1A (ATP synthase subunit alpha, mitochondrial) expression. ATP5F1A encodes a subunit of mitochondrial ATP synthase. Mitochondrial ATP synthase catalyzes ATP synthesis, using an electrochemical gradient of protons across the inner membrane during oxidative phosphorylation. ATP synthase is composed of two linked multi-subunit complexes: the soluble catalytic core, F1, and the membrane-spanning component, Fₒ, comprising the proton channel. The catalytic portion of mitochondrial ATP synthase consists of 5 different subunits (alpha, beta, gamma, delta, and epsilon) assembled with a stoichiometry of 3 alpha, 3 beta, and a single representative of the other 3. The proton channel consists of three main subunits (a, b, c). ATP5F1A encodes the alpha subunit of the catalytic core. Alternatively spliced transcript variants encoding the same protein have been identified.

In some embodiments, a patient treated using the subject methods exhibits an increase in UQCRC2 (Cytochrome b-c1 complex subunit 2, mitochondrial) expression. UQCRC2 encodes a protein that is part of the mitochondrial respiratory chain. The respiratory chain contains three multisubunit complexes succinate dehydrogenase (complex II, CII), ubiquinol-cytochrome c oxidoreductase (cytochrome b-c1 complex, complex III, CIII) and cytochrome c oxidase (complex IV, CIV), that cooperate to transfer electrons derived from NADH and succinate to molecular oxygen, creating an electrochemical gradient over the inner membrane that drives transmembrane transport and the ATP synthase. The cytochrome b-c1 complex catalyzes electron transfer from ubiquinol to cytochrome c, linking this redox reaction to translocation of protons across the mitochondrial inner membrane, with protons being carried across the membrane as hydrogens on the quinol. In the process called Q cycle, 2 protons are consumed from the matrix, 4 protons are released into the intermembrane space and 2 electrons are passed to cytochrome c (by similarity). The two core subunits UQCRC1/QCR1 and UQCRC2/QCR2 are homologous to the 2 mitochondrial-processing peptidase (MPP) subunits beta-MPP and alpha-MPP respectively, and they seem to have preserved their MPP processing properties.

In some embodiments, a patient treated using the subject methods exhibits an increase in MTCO1 (Cytochrome c oxidase subunit 1) expression. MTCO1 encodes for Cytochrome c oxidase subunit 1, which the last enzyme in the mitochondrial electron transport chain that drives oxidative phosphorylation. The respiratory chain contains 3 multisubunit complexes succinate dehydrogenase (complex II, CII), ubiquinol-cytochrome c oxidoreductase (cytochrome b-c1 complex, complex III, CIII) and cytochrome c oxidase (complex IV, CIV), that cooperate to transfer electrons derived from NADH and succinate to molecular oxygen, creating an electrochemical gradient over the inner membrane that drives transmembrane transport and the ATP synthase. Cytochrome c oxidase is the component of the respiratory chain that catalyzes the reduction of oxygen to water. Electrons originating from reduced cytochrome c in the intermembrane space (IMS) are transferred via the dinuclear copper A center (CU(A)) of subunit 2 and heme A of subunit 1 to the active site in subunit 1, a binuclear center (BNC) formed by heme A3 and copper B (CU(B)). The BNC reduces molecular oxygen to 2 water molecules using 4 electrons from cytochrome c in the IMS and 4 protons from the mitochondrial matrix.

In some embodiments, a patient treated using the subject methods exhibits an increase in NUDFB5 (NADH dehydrogenase [ubiquinone] 1 beta subcomplex subunit 5, mitochondrial) expression. NDUFB5 gene codes for a subunit of Complex I of the respiratory chain, which transfers electrons from NADH to ubiquinone.

### VII. Treatment Modalities

In the methods provided herein, therapy is used to provide a positive therapeutic response with respect to a disease or condition. By “positive therapeutic response” is intended a reduction in muscle fatigue and/or an improvement in endurance (e.g., muscle and/or aerobic endurance). In some embodiments, a positive therapeutic response includes a reduction of white adipose tissue in a patient. In some embodiments, a positive therapeutic response includes a reduction of glucose intolerance in a patient. In some embodiments, a positive therapeutic response includes an improvement in maximum aerobic capacity in the patient. In certain embodiments, a positive therapeutic response includes improved motor coordination in the patient. In some embodiments, a positive therapeutic response includes enhanced expression of one or more of the biomarkers described herein.

Treatment includes a “therapeutically effective amount” of the IL-6 medicaments used. A “therapeutically effective amount” refers to an amount effective, at dosages and for periods of time necessary, to achieve a desired therapeutic result (e.g., a positive therapeutic response as discussed herein). As provided herein, a therapeutically effective amount refers to an amount effective of IL-6, at dosages and for periods of time necessary, to achieve a desired therapeutic result in conjunction with an exercise training program. In some embodiments, a therapeutically effective amount is an amount that minimizes or reduces muscle fatigue in a patient in conjunction with the exercise training program. In certain embodiments, a therapeutically effective amount is an amount that improves muscle endurance or aerobic endurance in a patient in conjunction with an exercise training program. In some embodiments, a therapeutically effective amount is an amount that reduces white adipose tissue in the patient in conjunction with an exercise training program. In some embodiments, a therapeutically effective amount is an amount that reduces abdominal fat and/or visceral fat in the patient in conjunction with an exercise training program. In some embodiments, a therapeutically effective amount is an amount that reduces exercise-related lactic acidosis in a patient (blood lactate >5.0 mmol/L in combination with pH<7.35). In some embodiments, a therapeutically effective amount reduces age-related decline in peak oxygen uptake (\dot{V}O₂ₚₑₐₖ) in the patient. Such parameters can be measured using any suitable technique known in the art.

The metrics assessed to determine therapeutic effectiveness will depend on the exercises performed during the exercise training program. In some embodiments, the therapy improves muscle endurance in the individual. Muscle endurance is the ability of a muscle or a muscle group to exert force to overcome a resistance many times. In some embodiments, the measurement of muscle endurance is based on the number of repetitions performed. Muscle endurance is specific to the assessment. In some embodiments wherein the exercise training program includes endurance training, the patient can be assessed for the length of time the patient engaged in the endurance training for a particular session at a particular pace without rest. In some embodiments, wherein the exercise is resistance training, the number of repetitions without breaking form and the amount of resistance used is assessed. The measurements are recorded through the treatment program and improvements in performance of the particular exercise performed are monitored through the course of the treatment program.

In some embodiments, the methods provided herein reduce muscle fatigue in the individual after an exercise session. Muscle fatigue is a decrease in maximal voluntary contraction (MVC) torque/force/power that can be produced by a muscle group. Muscle fatigue can be measured using any suitable method. In some embodiments, muscle fatigue is measured using a dynamometer and/or a surface EMG. In some embodiments, muscle fatigue is measured as an increase in lactic acid (blood lactate >5.0 mmol/L in combination with pH<7.35) and/or reduction of muscular glycogen.

In some embodiments, the methods provided herein improve muscle recovery after an exercise session. Muscle recovery can be measured using any suitable method. In some embodiments, muscle recovery is measured using plasma creatine and/or creatine kinase levels. In some embodiments, muscle recovery is measured as a change in fiber type and infiltration of connective tissue and fat between muscle fibers, lean body mass (dual-energy X-ray absorptiometry) and/or quadriceps cross-sectional area (CSA; computed tomography). In recovery from exhaustion, muscle recovery is measured using lactate levels and peak oxygen uptake (VO₂peak), maximal strength.

In some embodiments, the methods provided herein improve aerobic endurance. In exemplary embodiments, the methods provided herein improve one or more aspects of aerobic endurance in the individual. Aspects of aerobic endurance include, but are not limited to, improvements in pulmonary function, cardiac function, circulation, microvasculature, oxygen extraction and use in skeletal muscle, muscle metabolism and/or muscle contractile function. Such aspects may be assessed using any suitable technique known in the art.

A therapeutically effective amount may vary according to factors such as the age, sex, and weight of the individual, and the ability of the medicaments to elicit a desired response in the individual. A therapeutically effective amount is also one in which any toxic or detrimental effects of the IL-6 composition are outweighed by the therapeutically beneficial effects.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the IL-6 used in the methods described herein depend on the condition to be treated and may be determined by the persons skilled in the art. In some embodiments, the IL-6 is administered at a dose of from about 0.01 μg/kg-about 10 μg/kg. In some embodiments, the IL-6 is administered at a dose of from about 0.01-about 0.05 μg/kg, about 0.05-about 0.10 μg/kg, about 0.10-about 0.50 μg/kg, about 0.05-about 1.0 μg/kg, about 1-about 2 μg/kg, about 2-about 3 μg/kg, about 3-about 4 μg/kg, about 4-about 5 μg/kg, about 5-about 6 μg/kg, about 6-about 7 μg/kg, about 7-about 8 μg/kg, about 8-about 9 μg/kg, or about 9-about 10 μg/kg. In exemplary embodiments, the dose is from about 0.05 μg/kg-about 3 μg/kg. In some embodiments, the IL-6 is administered at a dose of from about 0.2 μg/kg-about 1 μg/kg.

In some embodiments, the IL-6 used in the methods described herein is administered at a dose of from about 0.75 μg to 750 μg. In some embodiments, the IL-6 is administered at a dose of from about 1 μg-about 5 μg, about 5 μg-about 10 μg, about 10 μg-about 25 μg, about 25 μg-about 50 μg, about 50 μg-about 100 μg, about 100 μg-about 150 μg, about 150 μg-about 200 μg, about 200 μg-about 250 μg, about 250 μg-about 300 μg, about 300 μg-about 350 μg, about 350 μg-about 400 μg, about 400 μg-about 450 μg, about 450 μg-about 500 μg, about 550 μg-about 600 μg, about 600 μg-about 650 μg, about 650 μg-about 700 μg, or about 700 μg-about 750 μg.

In some embodiments, the IL-6 is administered as a weekly dose for a duration of the treatment. In some embodiments, the IL-6 is administered 1, 2, 3, 4, 5, 6, 7, 8, 9, or 10 times or more a week for the duration of the treatment. In some embodiments, the IL-6 is administered 1, 2, 3, 6, 12 or 24 hours or less prior to an exercise session of the exercise training program. In certain embodiments, the IL-6 is administered 5, 15, 30, 45 or 60 minutes or less prior to an exercise session. In some embodiments, the IL-6 is administered after an exercise session of the exercise training program. In some embodiments, the IL-6 is administered no more than 24 hours after an exercise session of the exercise training program. In some embodiments, the IL-6 is administered no more than 1, 2, 3, 6, 12 or 24 hours after an exercise session of the exercise training program. In some embodiments, the IL-6 is administered no more than 5, 15, 30, 45 or 60 minutes after an exercise session.

In some embodiments, the IL-6 compositions provided herein are administered 4 hours, 8 hours, 12 hours, 24 hours, 2 days, 3 days, 4 days, 5 days, 6 days, 1 week, 2 weeks, 3 weeks, one month, two months, three months, four months, five months, six months, seven months, eight months, nine months, ten months, eleven months, or a year apart.

A medical professional having ordinary skill in the art may readily determine and prescribe the effective amount of the pharmaceutical composition required. For example, a physician or a veterinarian could start doses of the medicament employed in the pharmaceutical composition at levels lower than that required in order to achieve the desired therapeutic effect and gradually increase the dosage until the desired effect is achieved.

In some embodiments, the IL-6 is co-administered with a therapeutic selected from vitamin D3, Resveratrol Setmelanotide, VIP, or an iron supplement. In some embodiments, the IL-6 is co-administered with one or more activators of the AMPK pathway. AMPK activators include, but are not limited to, resveratrol, aspirin, and metformin. In some embodiments, the IL-6 is administered with an activator of the adenylate cyclase pathway. Exemplary activators of the adenylate cyclase pathway include vasoactive intestinal peptide (VIP) and pituitary adenylate cyclase-activating polypeptide (PACAP). In some embodiments, the adenylate cyclase pathway activator is a melanocortin receptor activator. In some embodiments, the IL-6 is administered with an activator of NFR2 and/or Klotho expression (e.g., vitamin D3). In some embodiments, the IL-6 is administered with an inhibitor of a NFkB pro-inflammatory pathway. In certain embodiments, such inhibitor blocks TNF-α or IL-1 activity or their downstream signaling. In some embodiments, the method further comprises providing the subject with a free fatty acid supplement during and/or after an exercise session.

### Examples

Examples are provided below to illustrate the present invention. These examples are not meant to constrain the present invention to any particular application or theory of operation.

### A. Introduction

Aging is characterized by a progressive and inevitable loss of physiological integrity, leading to functional impairments and increased susceptibility to acute and chronic diseases [1]. While regular physical activity is considered to be an anthropological necessity for the maintenance of physiological function throughout life [2], a sedentary lifestyle is strongly associated with accelerated aging and reduced health span [3]. Even though skeletal muscle is a major driver of pathoetiology, deteriorations of the cardiorespiratory and neuromuscular systems with increasing age also play an important role in modulating the development of severe functional impairments and frailty, as they all contribute to a devastating vicious cycle. Initial decrements in physical performance, fatigue resistance and motor coordination result in unsteady gait and an increased perception of effort during activity, which leads to the avoidance of physical challenges and reduced voluntary exercise [4]. The ensuing muscular disuse further drives performance decline, the loss of muscle mass and strength, favors the development of chronic disorders and increases the risk of falls, disability and death [5]. Therefore, besides neurodegenerative events, deterioration in functional capacity of skeletal muscle is one of the major causes for loss of independence, admission to nursing homes, morbidity and mortality in the elderly. Of note, due to the high costs associated with care of these patients, sarcopenia and frailty put an escalating burden on health care systems [6, 7]. In light of an expanding global geriatric population [8], effective interventions to preserve or improve physical performance and functional capacities at advanced ages are thus becoming increasingly important, both on the individual, but also the societal levels.

Endurance exercise is an efficient way to improve cardiorespiratory, neuromuscular, and metabolic function, as it requires successful integration of these systems [9]. Endurance exercise training may thus provide a powerful countermeasure for muscular decline, loss of motor coordination and mobility, and metabolic deteriorations with aging [10-12]. Unfortunately however, several factors hamper the effectiveness of exercise-based interventions, in particular in the elderly. First, as inactivity and social commitments that require long sedentary periods emerged as common and widely accepted lifestyle choices in modern societies, acceptance of exercise-induced discomfort and pain as well as compliance with long-term training are major obstacles to implement exercise interventions in the human population, young and old. Second, at older age, muscle may show an attenuated immediate [13, 14] and/or an abbreviated response to mechanical loading and thus may require more intense and/or more frequent workouts to significantly adapt [16]. Third, aged muscle may be more vulnerable to exercise-induced injury compared with young muscle [17, 18] and display decreased regenerative capacity [19, 20], which can limit exercise intensity, the amount of training or the potential for improvement. Fourth, many elderly people have accumulated disuse complications and suffer from morbidity, frailty and coordinative deficits [21], which may not allow to manage or tolerate the strenuous or sustained training required to induce significant adaptations. Taken together, novel and effective strategies to leverage the beneficial effects of exercise training on physical performance and neuromuscular function in aging are highly desirable. However, effective and safe pharmacological therapies are lacking [22].

Myokines, signaling molecules produced and secreted by skeletal muscle, may play a pivotal role in meditating the positive muscular and systemic effects of exercise and adaptation to long-term training, by their auto-, para- and/or endocrine action [23, 24]. Interleukin-6 (IL-6), the founder member of the myokine family, increases up to 100-fold in the circulation in response to exercise, leading to a systemic spike towards the end of or shortly after a single bout of exercise, while returning back to baseline quickly thereafter [25]. The magnitude of the IL-6 response is proportional to the exercise duration and intensity, the amount of muscle mass activated, and depends on pre-exercise levels of muscle glycogen [26]. While skeletal muscle can both produce and secrete IL-6, neither its cellular origin during exercise (i.e. the contribution of skeletal muscle to the systemic peak), nor the mechanistic underpinnings of its muscular release have been formally established. Recently, it has been proposed that lactate production by the muscle on the verge of exhaustion may trigger IL-6 release from intramuscular storage vesicles (Hojman et al., 2019). Released IL-6 has been proposed to act as metabolic coordinator in the inter-organ crosstalk during exercise, by promoting endogenous hepatic glucose production [27, 28] and lipolysis in adipose tissue [29-32]. According to recent findings, IL-6 further facilitates the uptake and catabolismof energy substrates (i.e. glucose and fatty acids) in muscle fibers via a muscle-bone crosstalk and therefore, may be required for acute endurance exercise adaptation [33]. Whether and how IL-6 signaling during exercise training regulates long-term adaptation is however still unknown.

IL-6 is a strong pro-inflammatory cytokine important for an adequate immune response to infection, e.g. by activating immune cells, mounting the acute response in the liver or initiating the fever response in the hypothalamus [34]. Curiously, when elevated in the circulation during exercise, IL-6 exerts potent systemic anti-inflammatory effects [35]. Even though the exact mechanisms that differentiate pro- from anti-inflammatory action of IL-6 are not entirely elucidated, the timing and extent of IL-6 release, as well as the cellular and systemic contexts, might be important. A chronic, low-level increase in circulating IL-6, often closely mirrored by elevation of tumor necrosis factor alpha (TNF-α), interleukin-1 beta (IL-1B), and other pro-inflammatory cytokines, is observed in a number of chronic diseases [36]. In contrast, the transient elevation of higher concentrations of IL-6 after exercise is linked to a more anti-inflammatory environment, e.g. the elevation of IL-1 receptor antagonist that blocks IL-1β signaling and IL-10 that inhibits TNF-α production [37, 38]. This dichotomy in IL-6 functions results in differential therapeutic avenues for pathological contexts. Anti-IL-6 agents are tested or used in the treatment of diseases characterized by a persistent, sterile inflammation, such as type 2 diabetes, multiple sclerosis, atherosclerosis or rheumatoid arthritis. In comparison, the application of recombinant IL-6 (rIL-6) to mirror the anti-inflammatory, and hence potentially beneficial effects of IL-6 as a myokine, is still in its infancy for therapeutic use. For example, rIL-6-based therapy has been proposed to mitigate neuropathies associated with cancer chemotherapy and diabetes [40-42]. Surprisingly, the use of rIL-6 treatment mimicking the myokine function on age-associated decrements in physical performance and muscle function, and the combination with exercise to facilitate training and achieve synergistic effects, has not been studied so far.

Given the impaired exercise training capacity and/or blunted adaptive response of aged muscle to exercise stimuli, coupled with the proposed role of IL-6 in exercise adaptation, the aims of the current study were to investigate whether: (1) a moderate-intensity, low-volume 12-weeks endurance exercise regimen is sufficient to preserve or improve functional capacities in old mice, (2) the effect of the same intervention can be potentiated by exposing the animals to elevated IL-6 levels during training sessions and (3) long-term, pulsatile rIL-6 treatment can elicit endurance training-like effects in old sedentary mice in a safe and tolerable manner.

### B. Methods

### 1. Animals

Aged C57BL/6JRj mice were obtained from Janvier Labs (Le Genest-Saint-Isle, France) at an age of 19 months and then kept under a 12 h light to dark cycle (lights on at 06:00 am) at 23° C. in the animal facility of the Biozentrum (Basel, Switzerland) until the end of the study. Mice were housed single caged with enrichment and received ad libitum access to regular chow and water. All experiments were approved by the veterinary office of the canton Basel-Stadt (Switzerland) and performed in accordance with the Swiss federal guidelines for animal experimentation under consideration of the wellbeing of the animals and the 3R (replace, reduce, and refine) principle.

### 2. Study Design

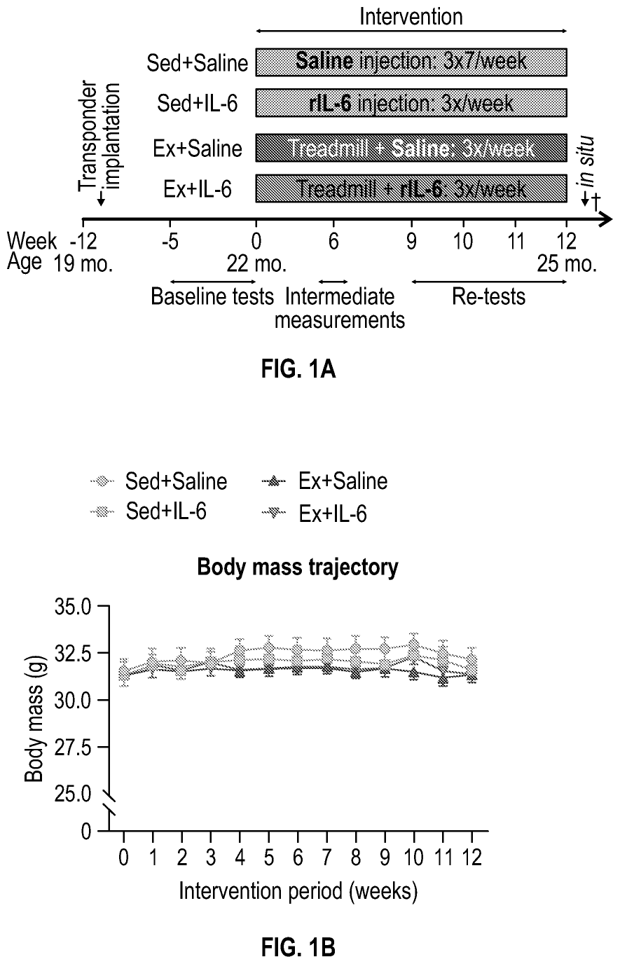

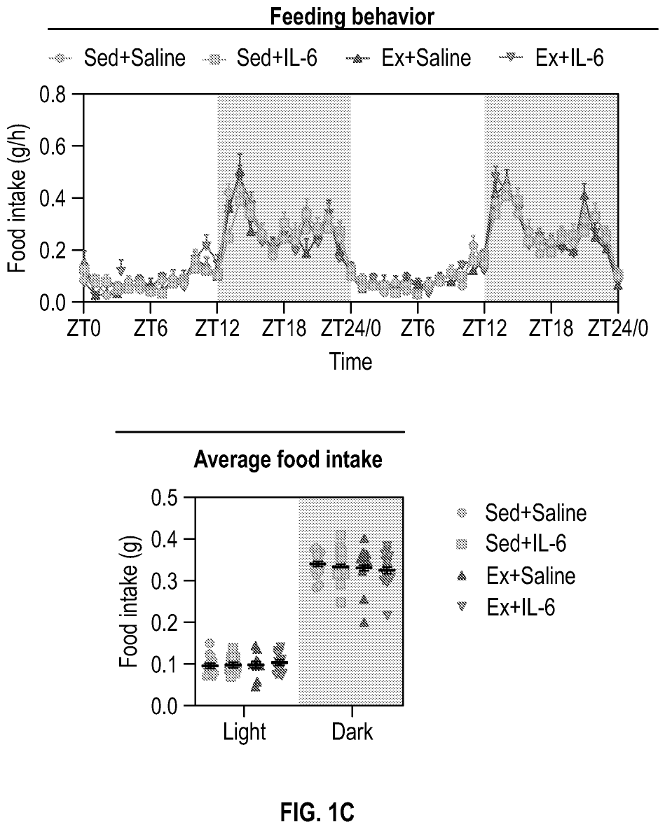

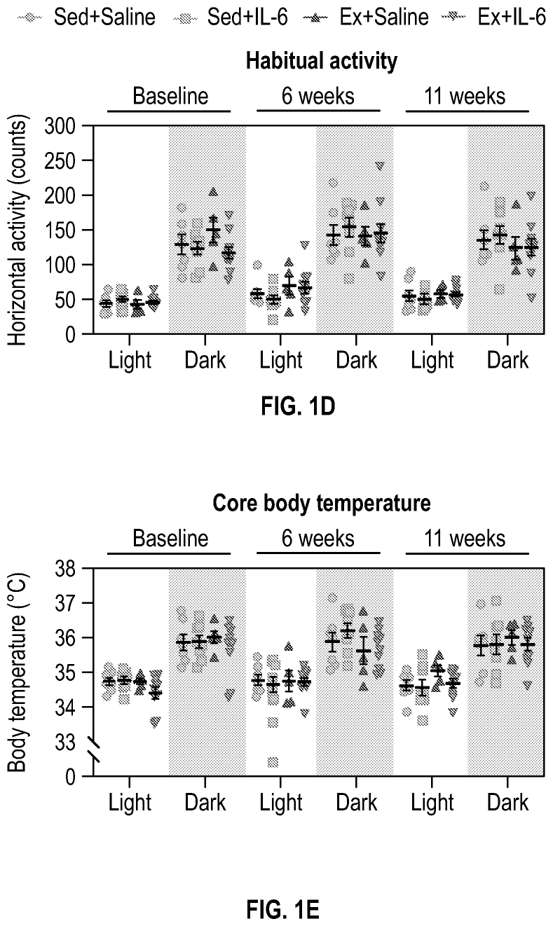

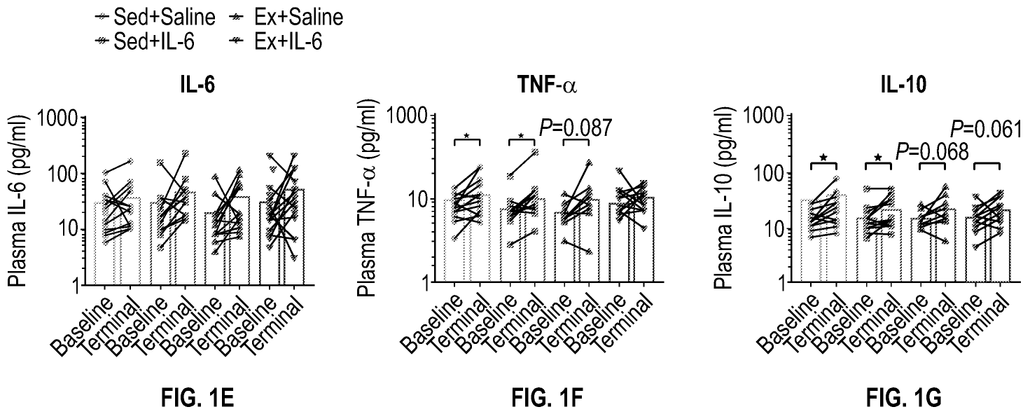

After initial acclimatization (at least 2 weeks) to the animal facility of the Biozentrum (Basel, Switzerland), subgroups of mice were implanted with a battery free transponder for the later application of a telemetric system to track spontaneous locomotor activity and core body temperature (FIG. 1A). In the weeks prior to the exercise and/or rIL-6 interventions, mice were subjected to a baseline characterization of behavior (e.g. spontaneous activity), gait (CatWalk analysis), motor coordination (Rotarod test), physical performance [e.g. running capacity and/or peak oxygen uptake (\dot{V}O₂ₚₑₐₖ)], metabolic parameters [e.g. body mass, body composition, rate of oxygen uptake (\dot{V}O₂) and carbon dioxide production (\dot{V}O₂), heat production, food intake, whole body glucose disposal] and systemic markers for metabolic and inflammatory status.

At 22 months of age, mice were divided into four experimental groups. Two groups of animals were kept under sedentary conditions, of which one was subcutaneously injected with low doses (10 μg/kg) of murine rIL-6 three times per week, to mimic the transient increase of IL-6 plasma levels observed in response to single endurance exercise bouts (Sed+IL-6) and the other was sham injected with saline solution (Sed+Saline). Two additional groups of animals were engaged in a moderate-intensity, low-volume treadmill exercise-training regimen, in one group paired with sham injections (Ex+Saline), and in the other group with rIL-6 administration (Ex+IL-6). All interventions lasted for a period of 12 weeks and until mice reached an age of 25 months in order to investigate the long-term effect of recurrent transient exposure to rIL-6 and/or exercise training with aging. Experimental groups were generated in a semi-randomized manner by first randomly assigning mice to one of four groups and then equating for baseline body mass and maximum distance achieved in the maximum running capacity test (long duration incremental step protocol). Of note, group equalization was performed with the initial number of animals before starting the rIL-6 and/or exercise treatment and therefore prior to any dropouts or deaths occurring during the intervention.

Habitual activity and body temperature were monitored regularly throughout the study whereas gait, motor coordination, endurance performance and metabolic parameters were retested during the last third of the study. Of note, retests were scheduled as close as possible towards the end of the study but had to be distributed over the last 3 weeks due to feasibility reasons and in order to minimize stress level. After 12 weeks, the function of the m. tibialis anterior was evaluated in situ and subsequently, mice were sacrificed to obtain terminal blood and to harvest limb muscles and other tissues. This was performed 48 h after the last injection and/or exercise session in order to minimize acute effects, as the goal of the current study was to assess long-term adaptation to training and/or IL-6 treatment. Of note, data presented in this study resulted from three independent cohorts and most of the tests and measurements were only performed in subgroups of animals.

### 3. Plasma Sampling and Analyses

Blood was drawn from the tail vein before (baseline) and after 12 weeks of the intervention in heparin coated tubes (Sarstedt). Tubes were centrifuged at 2000 g for 5 min at 4° C., supernatant transferred to a fresh tube, snap-frozen in liquid nitrogen and stored at −80° C. until use.

Plasma cytokines were measured with a V-PLEX Proinflammatory Panel 1 Mouse Kit (Meso Scale Discovery) customized for five analytes (INF-γ, IL-1B, IL-6, IL-10, TNF-α) using a 2-fold dilution and following the manufacturer's instructions.

Terminal plasma levels of alanine transaminase (ALT), aspartate transaminase (AST), albumin, lactate dehydrogenase (LDH), total cholesterol, low-density lipoprotein (LDL), high-density lipoprotein (HDL), triglycerides and lipase were measured using the cobas c 111 analyzer (Cobas®, Roche Diagnostics AG) using a 3-fold dilution in water and the manufacturer's test kits for the individual analytes.

### 4. Body Composition

Body composition was assessed with an EchoMRI-100 analyzer (EchoMRI Medical Systems). Mice were placed into a transparent measuring tube containing small holes for air exchange in the wall at the end of the tube. To minimize movement while the measurement is taking place, mice were minimally restrained by introducing another tube with a smaller diameter. The individual measurements took less than 2 min. Measurements were taken at baseline and the day before euthanasia and dissection.

5. Energy Homeostasis Measurements with the Comprehensive Laboratory Animal Monitoring System (CLAMS)

In brief, food intake, the rate of oxygen consumption (\dot{V}O₂) and carbon dioxide production (\dot{V}CO₂) and spontaneous movements were measured using the Comprehensive Laboratory Animal Monitoring System (CLAMS, Columbus Instruments) and Oxymax System (Columbus Instruments) for indirect calorimetry. After measuring body mass, each mouse was placed into a sealed chamber (21.3 cm×11.6 cm×11.5 cm) individually. Measurements were continuously taken for 94 h in an environment-controlled cabinet chamber set at 23° C. with a 12 h light to dark cycle (lights on at 06:00 am). During this time, food and water were provided to the animals through the feeding and drinking devices provided by the CLAMS system. The amount of food consumed by each animal was monitored through a precision balance installed under the chamber. A specific gas blend (Primary Standard Grade; 20.5% O₂ and 0.5% CO₂ in N2) was used to calibrate the span or gain of both the O₂ and CO₂ sensors of the Oxymax system before each experiment and flow was set to 0.6 l/m. \dot{V} O₂ and \dot{V} CO₂ samplings were done sequentially in a 15 min interval. RER was calculated as \dot{V}CO₂/\dot{V}O̅₂.

### 6. Glucose Homeostasis

Mice were acclimatized to restraining a few days before the baseline measurement (i.e. before the exercise training and/or IL-6 interventions) of glucose homeostasis in order to reduce the stress level during the experiment. Mice were fasted 12 h overnight before intraperitoneal injection of a glucose solution diluted in NaCl (2 g glucose/kg body mass). Blood glucose levels were recorded from the tail vein with a glucose meter (Accu-Chek, Roche Diagnostics) at 0, 15, 30, 45, 60, 90 and 120 min after glucose injection. Area under the curve (AUC) was determined as incremental area (i.e., above baseline). The same procedure was repeated 9 weeks after the start of the intervention.

### 7. Body Temperature and Locomotor Activity Recordings

General locomotor activity and core body temperature data were acquired with the E-Mitter Telemetry System (STARR, Life Sciences Corp.) from animals placed with their home-cages in an environment-controlled cabinet (UniProtect Air Flow Cabinet, Bioscape). In short, small transponders (G2 E-Mitter, 15.5 mm×6.5 mm, 1.1 g; STARR, Life Sciences Corp.) were implanted into the abdominal cavity of the mice under isoflurane anesthesia (2% isoflurane+O₂). Mice were treated with Meloxicam (1 mg/kg) pre- and post-operatively and allowed to recover for at least three weeks before the first measurement. Horizontal activity and core body temperature was recorded with a PC-based acquisition system connected to ER4000 Receivers (VitalView, STARR Life Sciences Corp.) for 3 to 4 day periods.

### 8. Gait Analysis

The Noldus CatWalk XT system was used according to the CatWalk XT 10.6 Reference Manual and as previously described [43]. Before the baseline measurement, mice were habituated to the darkened experimental room and trained to cross the illuminated walkway for three consecutive days (with three trials per day). On the fourth day, each animal was tested by being placed at one extremity of the glass plate and being allowed to move freely back and forth the walkway. Run duration was between 0.5 and 5 s with a maximum allowed speed variation of 60% to be considered as successful. The minimum number of compliant runs to acquire was set to three. The camera gain was adjusted to 20 dB and the detection threshold to 0.1 with a red ceiling and a green walkway light of 17.7 V and 16.5 V, respectively. The same test procedure was applied during week 12 of the intervention, but without acclimatization. Runs recorded on the testing days were manually controlled for the following compliance criteria: runs were considered compliant if the animal walked continuously and straight, i.e. without turning the head to the side. One to three trials were used for the final analyses. Runs were analyzed by an independent investigator using the CatWalk XT software which measures diverse gait parameters such as those reported in this study i.e., body speed (cm/s), cadence (number of steps/s), stride length (distance between successive placements of the same paw in cm), stand time (s) and swing speed (speed of the paw during swings, which is the duration in seconds of no contact of a paw with the glass plate), step cycle (s), base of support (distance between paws of fore or hind limbs in cm).

### 9. Evaluation of Maximum Running Capacity (Treadmill Exhaustion Test)

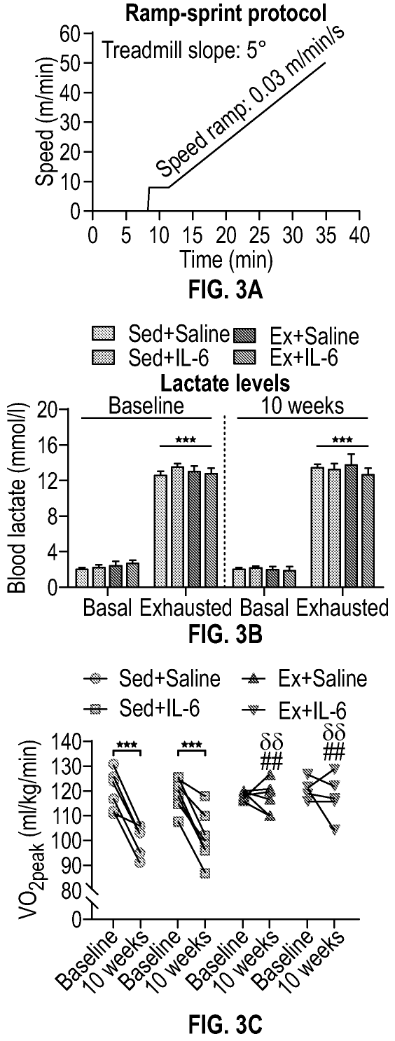

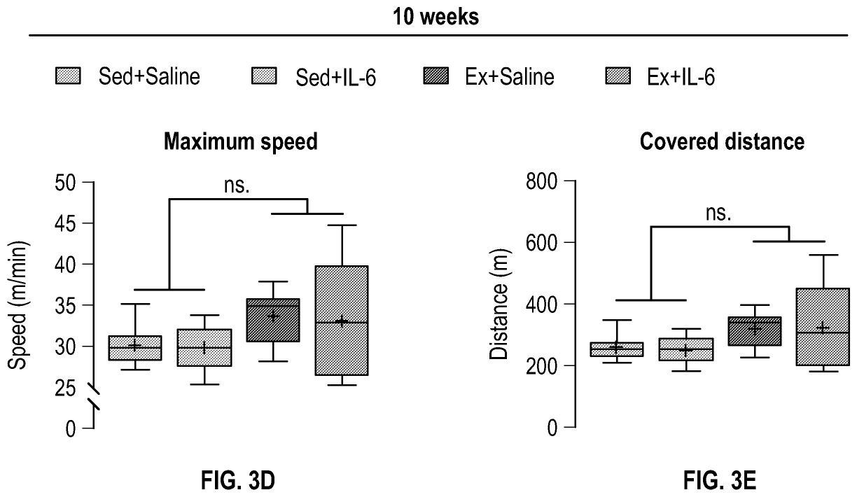

The running capacity test was performed on an open treadmill system (Columbus Instruments) with six lanes. Before the baseline test, mice were subject to a two-day acclimatization procedure. Day 1: mice were placed on the static treadmill band for 5 min followed by running at 5, 8 and 10 m/min for 5 min each at 5° inclination. Day 2: running at 5, 8, 10 and 12 m/min for 5 min each at 5° inclination. On the testing day (at least one day after the last acclimatization), mice were weighed and basal values for blood lactate were measured. Maximum running capacity was then evaluated by letting the mice run to exhaustion on an open treadmill with 5° inclination using a long duration incremental step protocol: 5 min at 5 m/min, 5 min at 8 m/min, 15 min at 10 m/min, followed by an increase of 2 m/min every 15 min until exhaustion (FIG. 3A). Exhaustion was determined by the animal failing to remain on the treadmill belt despite a mild electric stimulus (0.5 mA, 200 ms pulse, 1 Hz) and prodding. At this point, the mouse was taken off the treadmill to determine blood lactate levels immediately. Maximum running capacity was estimated using four parameters: the distance ran (m), the duration of the run (min), Peak power [Ppeak (W)] reached and total work [Wtot (J)] performed. Power was calculated as Ppeak=body mass (kg)·gravity (9.81 m/s2)·vertical speed (m/s·angle), and total work as Wtot (J)=body mass (kg) gravity (9.81 m/s2)·vertical speed (m/s·angle)·time (s).

Mice of all cohorts were retested with the same protocol during week 12 of the intervention. Tests were performed in the early morning (lights on) and at least 48 h after the last training session and/or IL-6 injection to minimize acute effects of the training and/or injections. For the test, mice were randomly assigned to groups of 5 to 6 animals and the testers were blinded for their intervention group assignment.

### 10. Ramp-Sprint Test for \dot{V}O₂Peak Measurements

Mice were acclimatized to the Metabolic Modular Treadmill (Columbus Instruments) set to 5° slope on three consecutive days (in the early morning, lights on) prior to the baseline measurement. The accommodation period consisted of: Day 1, placing the mice in the treadmill for 10 min without speed followed by 5 min at 5 m/min; Day 2, running at 5 m/min, 7 m/min and 10 m/min for 5 min each; Day 3, running for 8 m/min, 10 m/min and 12 m/min for 5 min each. After one resting day, a short and high-intensity ramp-sprint test was performed in the early morning using a protocol optimized to measure \dot{V}O₂ₚₑₐₖ [44]: After an 8 min resting-measurement followed by a 3 min warm-up at 8 m/min, speed was continuously increased by 0.03 m/min/s (i.e. slowly ramped up). Slope was set to 5°. Stainless steel grids at the end of each lane provided a mild electrical stimulus (0.5 mA, 200 ms pulse, 1 Hz) to keep the mice running. Mice ran until \dot{V}O₂ and RER values plateaued and/or if the animal remained on the electrical grid for more than 5 s without any attempt to go back on the treadmill. Before being placed on the treadmill, mice were weighed and tail blood lactate (Lactate Plus meter, Nova Biomedical) values were determined. Lactate values were measured again immediately after mice met the above described abortion criteria. A retest using the same protocol was performed during week 10 of the intervention. For the tests, mice were randomly assigned to groups of two animals and the operator was blinded for their intervention group assignment. A specific gas blend (Primary Standard Grade; 20.5% O₂ and 0.5% CO₂ in N₂) was used to calibrate the span or gain of both the O₂ and CO₂ sensors of the Oxymax system before each test session. Flow was set to 0.6 l/min. \dot{V}O₂ and \dot{V}CO₂ were measured every 5 s. RER was calculated as \dot{V}CO₂/\dot{V}O₂.

11. Treadmill Exercise Training and Saline or rIL-6 Injections

Mice were trained 3 times per week on an open treadmill (Clumbus Instruments) in the early morning (between 06:00 am and 09:30 am, lights on). Training intensity was determined based on the average peak speed (vpeak) reached during the maximum running capacity test (long duration incremental step protocol). Maximum speed was 50% vpeak in the first training session and was gradually increased every following session until 80% vpeak, which was reached after 5 weeks. In each training session, the speed was slowly ramped up during the first 5-10 min up to the scheduled maximum speed of the given session. Mice were encouraged to run continuously on the belt without resting by gentle prodding. Training sessions lasted 45 to 50 min until all mice covered the same total distance. Mice were injected with a recombinant mouse IL-6 protein (406-ML-200/CF, R&D Systems) diluted in PBS (Dulbecco's Phosphate Buffered Saline, Sigma) to a dose of 10 μg/kg per injection (injection volume: 250-350 μl/mouse). Sham-treated animals were injected with PBS (Dulbecco's Phosphate Buffered Saline, Sigma) at volumes that would correspond to rIL-6 dilutions. All mice were injected three times per week in the early morning (lights on) and exercising mice received their injection 30 min prior to each training session to expose them to elevated IL-6 levels during and following the exercise bouts.

### 12. Evaluation of Muscle Function In Situ

Function of the m. tibialis anterior was evaluated in situ using the 1300A Whole-Animal System (Aurora Scientific) as previously described [43, 45]. In brief, mice were anesthetized using pentobarbital (Esconarkon; 90-150 mg/kg) and kept on a heating pad during the whole procedure. The hind limb skin was carefully removed under a microscope to expose the m. tibialis anterior. The distal tendon was freed from connective tissue, tied with square knots, then released and attached to the mechanical force transducer. The knee was immobilized by passing a stainless steel pin or syringe needle behind the patellar tendon. Stimulation electrodes were placed underneath the sciatic nerve, which was previously exposed at the hip. Exposed tendon, nerve and muscle were constantly kept moist with pre-warmed mineral oil (Sigma). In all experiments, 0.2 s pulses at 15 V were used and the muscle was allowed to rest after each stimulation. Prior to twitch and tetanic force measurements, two 100 Hz tetanic stimulations were applied to tighten the knots. Muscle optimal length (LO) was then determined by applying successive single-twitch stimulations and adjusting basal muscle tension until maximal isometric twitch force (Pt). The muscle was then stimulated at different frequencies to obtain absolute maximal tetanic force. To test fatigue resistance, the m. tibialis anterior was subjected to a series of successive 100 Hz tetanic stimulations delivered every 2 s for 4 min. Recovery from fatigue was assessed after 1, 2 and 3 min with a single 100 Hz tetanic stimulation. Muscle mass and Lo were used to determine cross-sectional area to obtain specific isometric twitch force (sPt; kN/mm2) and tetanic force (sPO; kN/mm2). Animals were euthanized at the end of the experiment by thoracic opening and by drawing terminal blood via heart puncture.

### 13. Grip Strength In Vivo

Muscle force was estimated in vivo by measuring peak force of whole limb (i.e. fore and hind limbs together) grip using a Grip Strength Meter (Chatillon; Columbus Instruments) after 11 weeks. In brief, a mouse was gripped by the base of its tail between the thumb and the forefinger and placed with all four limbs on the angled mesh pull bar assembly connected to the force transducer. When the mouse was grasping properly with all four limbs, it was pulled along a straight line leading away from the sensor until the grip was released. A total of four trials were performed with at least 10 min break between trials. Each trial consisted of a series of four pulls with a short latency between each pull, where the highest force attained during the pulls was recorded in kilogram-force (kgf; one kgf is equal to 9.806650 N). The median of all four trials was normalized to body mass.

### 14. Assessment of Motor Coordination (Rotarod Test)

Mice were familiarized with the Rotarod instrument (Ugo basile, Model 47600) and taught to walk straight ahead without turning around on three consecutive days, by performing three 3 min trials in the fix mode with a constant speed of 4, 8 and 12 rpm on each day. On the test day, the Rotarod was set to acceleration mode (4 to 40 rpm over 5 min). Each mouse performed four consecutive test trials (with at least 10 min break between trials) and the time (s) as well as the speed (rpm) at which a mouse fell was recorded. Animals showing noncompliant behavior i.e. animals clinging to the rod and completing passive rotations were not included into the analysis. The median of trials longer than 100 s was taken as readout. Mice were tested at baseline (before the intervention) and during week 11 of the intervention.

### 15. Total Muscle RNA Extraction and Quantitative Real-Time PCR

Total RNA was extracted from the m. quadriceps femoris of the unstimulated (in situ) left leg using a hybrid method as previously described [43]. In brief, TRI-Reagent (Sigma) was combined with on-column RNA purification with DNase treatment (RNease Mini Kit, Qiagen). RNA quantity and purity was evaluated using the NanoDrop OneC spectrophotometer (Thermo Scientific; A260/A280 and A260/A230) values were >1.9) and 1 μg of total RNA was reverse transcribed using the High Capacity cDNA RT Kit (Applied Biosystems). Quantitative real-time PCR was performed with Fast SYBR Green (Applied Biosystems) using the QuantStudio Real-Time PCR System (Applied Biosystems). PCR reactions were done in duplicate with the addition of negative controls (i.e., no reverse transcription and no template controls). Relative gene expression levels were determined using the comparative ΔΔCT method to normalize target gene mRNA to Tbp and to the average of the control group (Sed+Saline). Primer sequences are summarized in Table 1.

TABLE S1
Primer sequences used for qPCR

| Gene | | |
|---|---|---|
| name | Forward primer | Reverse primer |
| Acc1 | AAGGCTATGTGAAGGATG | CTGTCTGAAGAGGTTAGG |
| Acc2 | GGGCTCGGGCATGATTG | CAGGTAAGCCCCGATTCCA |
| Atp5 | TCTCCATGCCTCTAACACTCG | CCAGGTCAACAGACGTGTCAG |
| Atp6 | ACTATGAGCTGGAGCCGTAATTACA | TGGAAGGAAGTGGGCAAGTG |
| Cd36 | AAGAGGTCCTTACACATACAGAGTTC | AGCTGCTACAGCCAGATTCA |
| Cptlb | GCACACCAGGCAGTAGCTTT | CAGGAGTTGATTCCAGACAGGA |
| Cox1 | TGCTAGCCGCAGGCATTACT | GCGGGATCAAAGAAAGTTGTG |
| Cs | CCCAGGATACGGTCATGCA | GCAAACTCTCGCTGACAGGAA |
| Cyc | ACAAGAAGACTCAAATGTGTTTCAGTT | TGCACTGTCAAGAATAGACAGTTG |
| | T | C |
| Fh1 | TGCTCTCAGTGCAAAATCCAA | CGTGTGAGTTCGCCCAATT |
| Glut4 | CATGGCTGTCGCTGGTTTC | AAACCCATGCCGACAATGA |
| ^{G}yg¹ | CGGCCACACTATGACAGATCA | TGTGGGCTGGTGAGTACAAC |
| Gys1 | TACACTGTGCTGCAGACGAA | CGCCCAAAATACACCTTACAAC |
| Hk2 | CCCTGCCACCAGACGAAA | GACTTGAACCCCTTAGTCCATGA |
| Mlycd | ACTCCATCAGCCTGACCCAG | ACCCCTTGAGGCTCTCGTGA |
| Ndufa5 | ACATGCAGCCTATAGAAAATACACAG | TCCGCCTTGACCATATCCA |
| | A | |
| Ndufb5 | TTTTCTCACGCGGAGCTTTC | TGCCATGGTCCCCACTGT |
| Ndufb8 | CAAGAAGTATAACATGCGAGTGGAA | CCATACCCCATGCCATCATC |
| Pdha | GAAGATGCTTGCCGCTGTATC | CCGATGAAGGTCACATTTCTTAAT |
| Pdk4 | AAAATTTCCAGGCCAACCAA | CGAAGAGCATGTGGTGAAGGT |
| Pdp1 | CGGGCACTGCTACCTATCCTT | ACAATTTGGACGCCTCCTTACT |
| Pfkm | TGTGGTCCGAGTTGGTATCTT | GCACTTCCAATCACTGTGCC |
| Pygm | GGTTTATGGTGCCGAGGACT | GGCGGCGGGAATAACTTTCT |
| Pgm1 | AAAAATCAGGGCTTGCGGC | TGGCAACGTCCTTCTCGTAG |
| Sdha | GCTGGTGTGGATGTCACTAAGG | CCCACCCATGTTGTAATGCA |
| Tbc1d1 | CATAAAGAACACACTCCCCAACCT | TGCTTGGCGATGTCCATCT |
| Tbc1d4 | GTACCGACCGGATATGATGTCA | CGGTGGTAGTCATGAAGGAGTCT |
| Tbp | TGCTGTTGGTGATTGTTGGT | CTGGCTTGTGTGGGAAAGAT |
| Uqcrc2 | CCCATCTTGCTTTGCTGTCTG | AATAAAATCTCGAGAAGGACCCG |

### 16. Protein Extraction and Immunoblotting

Equal amounts (30 mg) of frozen powdered tissue of the unstimulated (in situ) m. quadriceps femoris was homogenized in 300 μl of ice cold RIPA buffer [150 mM NaCl, 1% v/v Nonidet-P40 substitute, 0.2% v/v Na-deoxycholate, 0.1% v/v SDS, 50 mM TRIS-HCl (pH 7.5), 1 mM EDTA, 0.5 mM EGTA, 1 mM DTT, 10 mM Nicotinamide] containing freshly added protease inhibitors (complete mini EDTA free, Roche) and phosphatase inhibitors (PhosStop, Roche) using a bead homogenizer and metal beads [Qiagen, stainless steel 5 mm (200 pc)] and incubated for 1 h at 4° C. Next, tubes were centrifuged at 16′000 g for 10 min at 4° C. and the supernatant was transferred to a fresh tube. Protein concentration was measured with the Bradford protein assay (Quick Start Bradford 1× Dye reagent, BioRad) and BSA standards (Pierce). Samples were diluted in RIPA buffer and 4× Laemmli Sample Buffer (BioRad) with 2-Mercaptoethanol (Sigma) and boiled for 5 min at 95° C. or at 50° C. for OXPHOS blots. Equal amounts of protein were separated on 4-20% Mini-Protean TGX Precast Protein Gels (BioRad) and transferred on Nitrocellulose membranes (Amersham Protran 0.45 NC, 10600007, 0-45 μm). Membranes were blocked for 1 h in 5% milk in Tris-buffered saline+0.1% Tween 20 (TBS-T) before overnight incubation at 4° C. and shaking with the following antibodies: LDH-H (1:5000 in 5% BSA, TBS-T, NB110-57160, NOVUS Biologicals), Sarcomeric α-Actinin (1:5000 in 5% BSA, TBS-T, A7732, Sigma-Aldrich), Total OXPHOS Rodent WB Antibody Cocktail (1:1000 in 1% milk, PBS, ab110413, Abcam), eEF2 (1:1000 in 5% BSA, TBS-T #2332, Cell Signaling), NDUFB5 (1:500 in 5% BSA, TBS-T, #PA5-98000, Invitrogen Thermo Fisher Scientific), PDK4 P-24 (1:500 in 5% BSA, TBS-T, sc-120841, Santa Cruz), STAT3 (1:1000 in 5% milk, TBS-T, #9139, Cell Signaling). After washing, membranes were incubated with peroxidase-conjugated secondary antibodies (1:10′000 in TBS-T, Dako) for 1 h at room temperature, followed by antibody binding detection using chemiluminescence horseradish peroxidase substrate detection kits (ECL, Supersignal West Dura or Femto; Pierce) and a Fusion FX imager (Viliber). Quantification was done with the Fusion FX software. Relative protein levels were determined by normalizing the band intensity of the target to the loading control (α-Actinin, eEF2 or Ponceau S stain) and to the average of the control group (Sed+Saline) within one gel.

### 17. Statistical Analyses

Data represent either mean values±standard error of the mean (SEM) including individual values where possible, or individual points where paired values are connected with a black line, or are presented as box and whisker plots. In the latter, boxes depict the 25th and 75th percentiles (upper and lower perimeters), the median (midline) and the mean (cross). Whiskers are plotted based on the interquartile range (IQR) i.e. the difference between the 25th and 75th percentiles. The upper whisker is drawn to the largest value in the data set that is smaller than (or equal to) the 75th percentile plus 1.5 times IQR (upper fence) and the lower whisker is drawn to the smallest value in the data set that is greater than (or equal to) the 25th percentile minus 1.5 times IQR (lower fence). Any values greater than the upper or smaller than the lower fence are plotted as individual points. Data were represented and analyzed using the GraphPad Prism 8.0 software. The n number used for each experiment is indicated in the figure legends. Within group comparisons of baseline test and re-test measurements were performed with a paired Student's t-test. For between group comparisons, either one-way or two-way ANOVA was performed followed by Sidak's multiple comparisons as post-hoc test if the interaction term was significant. Dropout curves of the distance ran (m) during the maximum running capacity test were plotted in the form of “survival curves” and were compared using a logrank test for trend. This test is using chi-square to compute a P value by testing the null hypothesis that there is no linear trend between column order (i.e. the order of plotted curves from left to right) and survival (i.e. keeping on running). The level of significance was set at P<0.05 for all statistical tests.

### C. Results

To study the endurance exercise adaptive response in old aging mice and to test whether rIL-6 can act as a therapeutic agent to induce exercise-like effects or to potentiate long-term exercise adaptation, we used aged C57BL/6JRj mice as an experimental model. A schematic overview showing the timeline and experimental design is depicted in FIG. 1A. After a baseline characterization, mice were divided into four experimental groups. Two groups of animals were kept under sedentary conditions, of which one was subcutaneously injected with low doses (10 μg/kg) of rIL-6 protein three times per week, to mimic the transient increase of IL-6 plasma levels observed in response to single endurance exercise bouts. Two additional groups of animals were engaged in a moderate-intensity low-volume treadmill exercise-training regimen, in one group as a single intervention, in the other group combined with rIL-6 administration to enhance systemic IL-6 levels during and shortly after each training bout. The two groups that did not receive the rIL-6 treatment were sham injected with saline solution.

Long-term rIL-6 treatment alone or in combination with exercise is safe and well tolerated. Epidemiological evidence suggests a link between IL-6 and age-related diseases associated with chronic systemic low-grade inflammation [46], while there is a general lack of data regarding the safety of long-term recurrent rIL-6 administration. We therefore first monitored general behavior and wellbeing and assessed metabolic and inflammatory profiles under habitual non-exercise conditions.

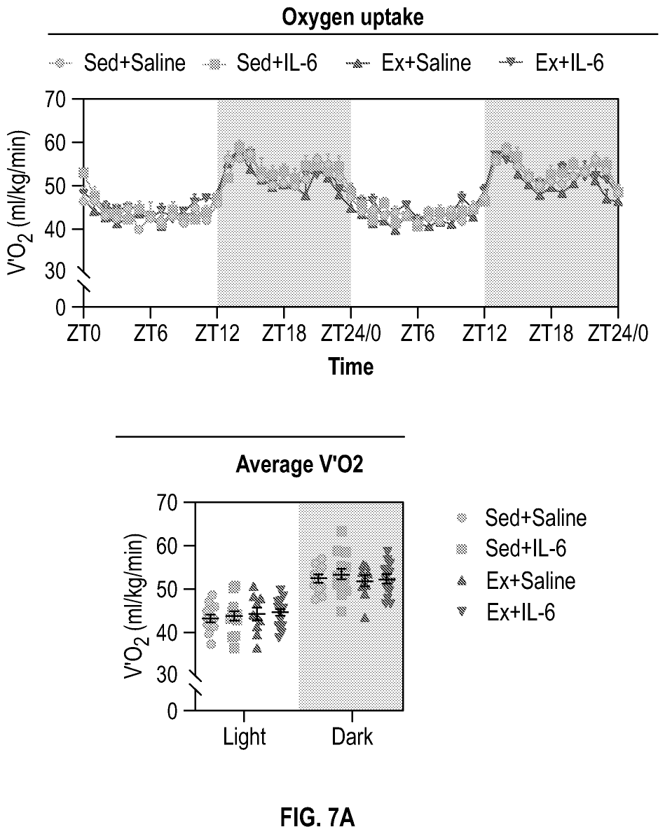

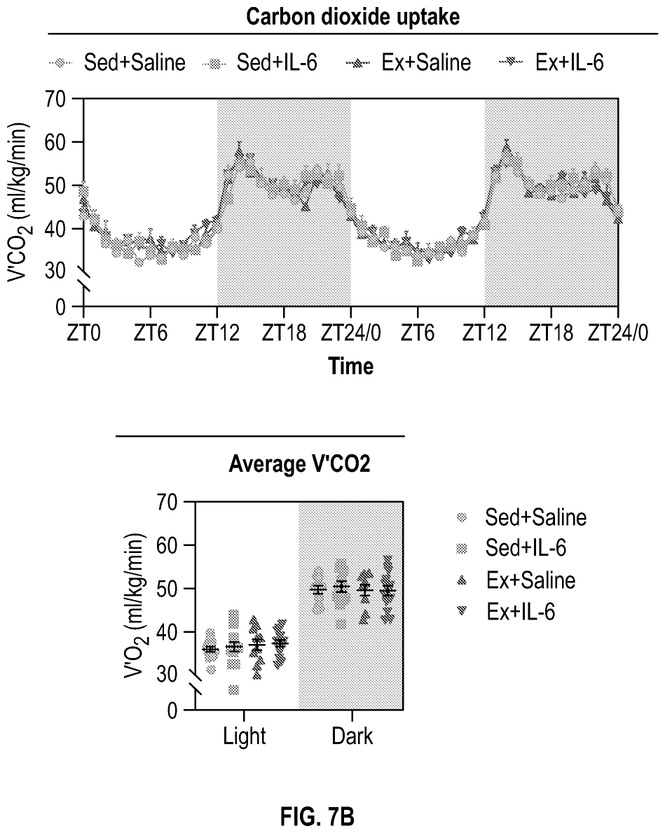

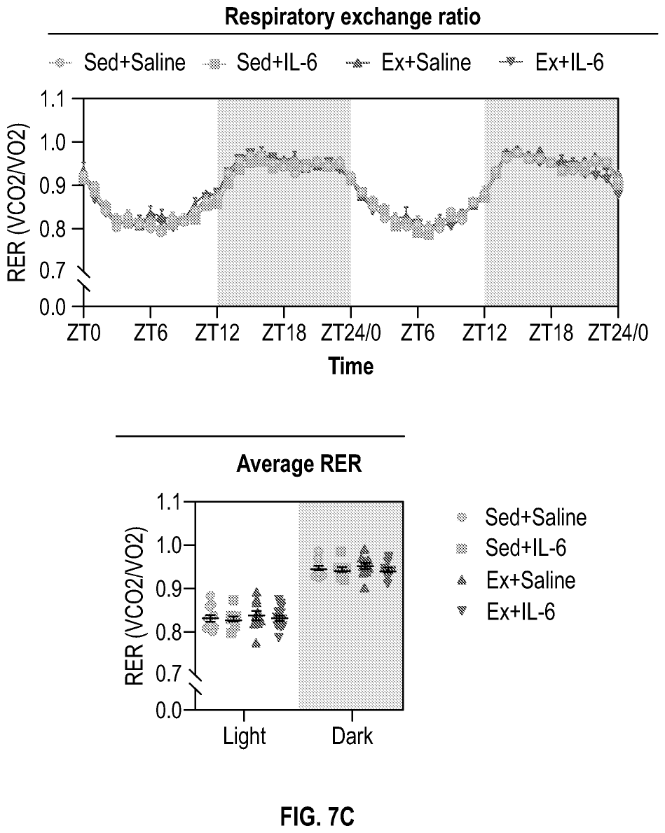

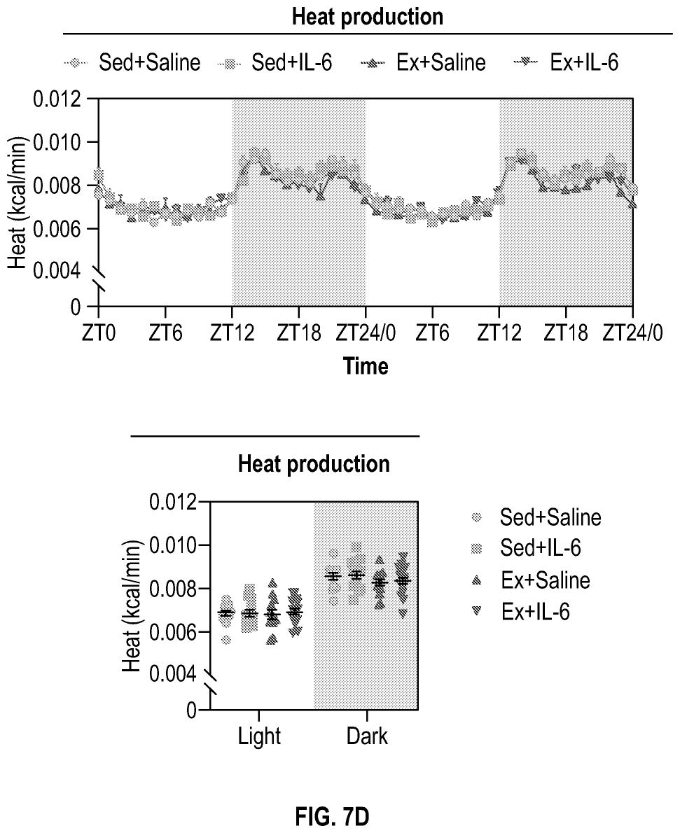

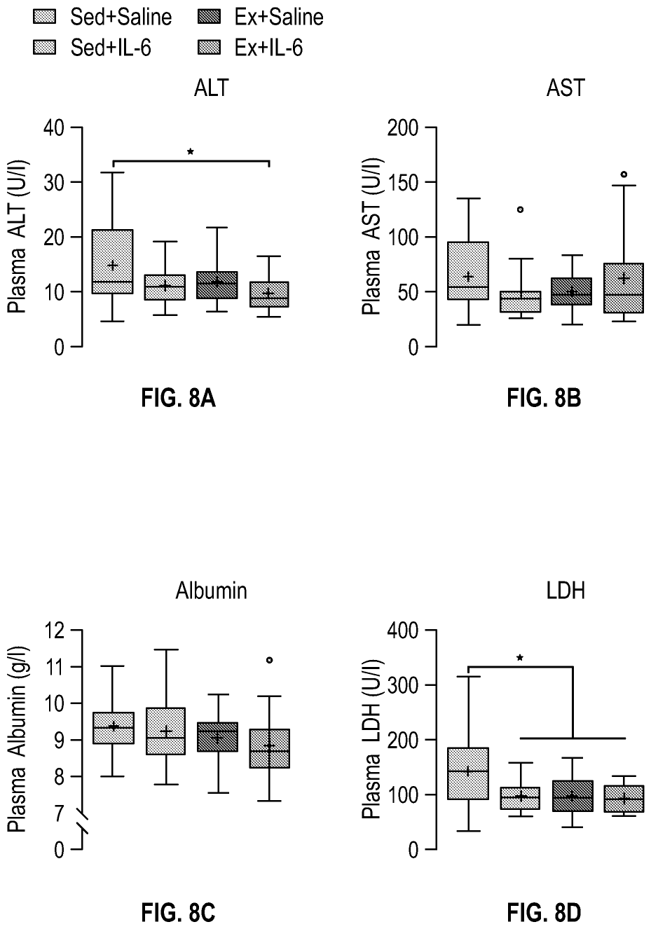

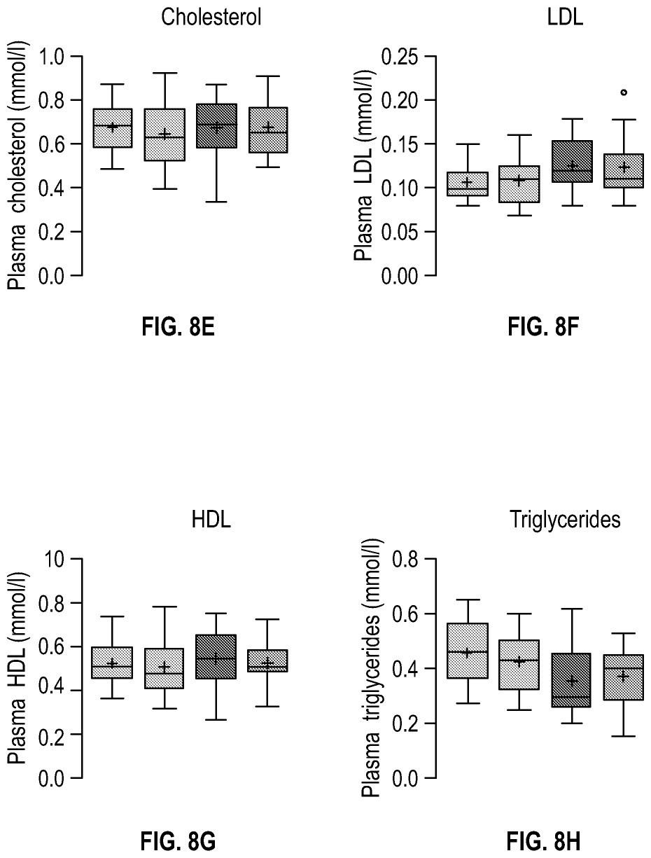

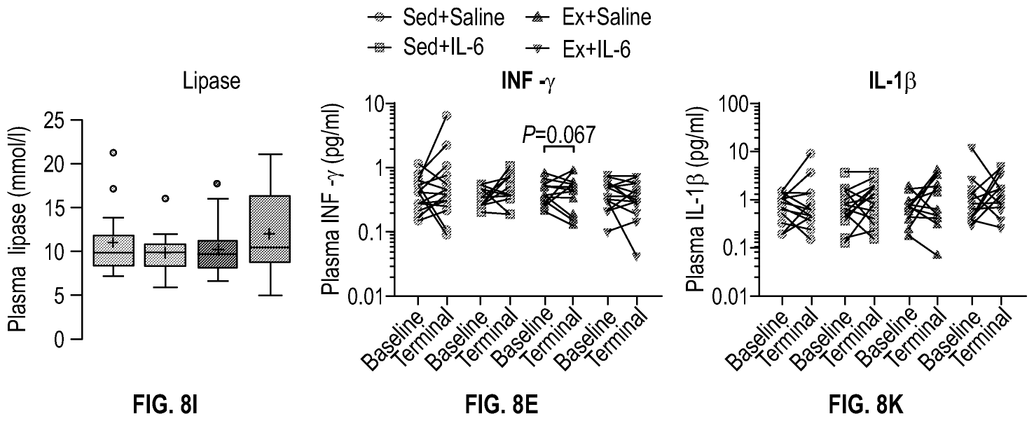

All four groups showed similar body mass trajectories throughout the study, and there was no difference between groups at any of the individual time points (FIG. 1B). Comprehensive analysis of feeding behavior (FIG. 1C), habitual activity (FIG. 1D) as well as several metabolic parameters [rate of oxygen consumption (\dot{V}O₂), carbon dioxide production (\dot{V}CO₂), respiratory exchange ratio (RER) and heat production; FIG. 7A-D] revealed no significant differences between groups. In line with these observations, terminal plasma analyses of a suite of markers of blood biochemistry revealed no obvious metabolic changes or adverse effects of long-term rIL-6 treatment (FIG. 8A-G). ALT and AST, two common markers to assess liver toxicity, were either unchanged, or in the case of ALT, even reduced in the Ex+IL-6 group. Moreover, LDH was lowered by rIL-6 and/or training indicating reduced cellular damage in these mice. Because IL-6 has been implicated in fever-generation upon peripheral immune challenge and inflammation [47, 48], we implanted small transponders (G2 E-Mitter, Starr Life Sciences) into the abdominal cavity of mice to tightly monitor core body temperature throughout the study. Animals treated with rIL-6 did not display higher body temperatures compared to saline treated animals, suggesting that repeated rIL-6 injections did not elicit persistent pyrogenic effects (FIG. 1E). To gain further insights into whether rIL-6 treatment and/or exercise affected the inflammatory status of the animals, we measured cytokines in the plasma at baseline and after 12 weeks of treatment. Resting IL-6 levels remained constant in all four groups during the intervention and thus were not affected by regular rIL-6 administration (FIG. 1E). While interferon gamma (INF-γ) and IL-1β levels remained also unchanged (FIGS. 8J and K), TNF-α and IL-10 plasma concentrations increased significantly with age, but only in the sedentary groups (FIGS. 1F and G).

Finally, we did not observe an increase in adverse events or mortality related to rIL-6 treatment. On the contrary, only one mouse in the rIL-6 treated groups (sedentary and exercised) died during the intervention, while seven animals died in the saline treated groups (two in the sedentary and five in the exercise group). Four mice were excluded from the final analysis because of reasons unrelated to their treatment: two because of limb joint problems, one because of dermatitis (and elevated inflammatory markers already from baseline) and one because of a lung tumor revealed upon dissection.

1. Moderate-Intensity Low-Volume Endurance Training Preserves Glucose Tolerance with Aging

Aging is associated with deteriorations of glucose tolerance, while endurance exercise can improve metabolic homeostasis. Moreover, in a previous study with young mice, elevated IL-6 concentrations in response to exercise, as well as acute and short-term rIL-6 treatment, increased insulin secretion and glucose tolerance [49]. We therefore tested whether moderate endurance training and/or long-term pulsatile rIL-6 treatment affected the response to a glucose tolerance test (GTT) in old aging mice.

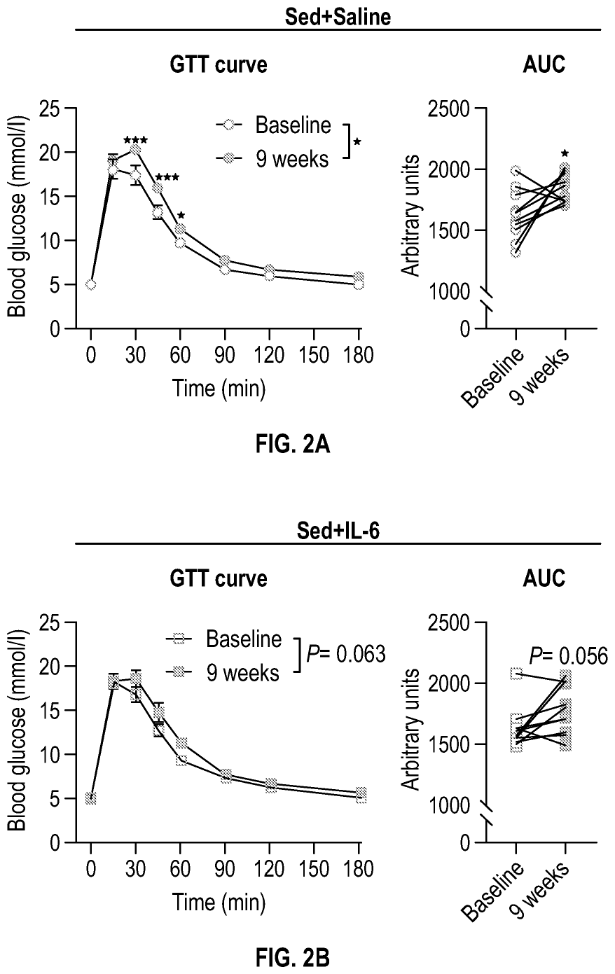

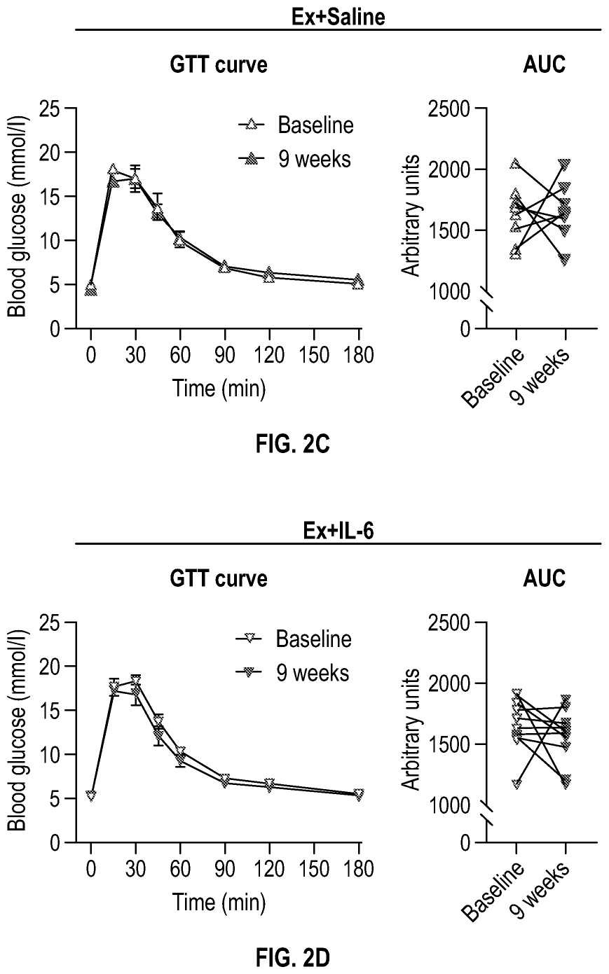

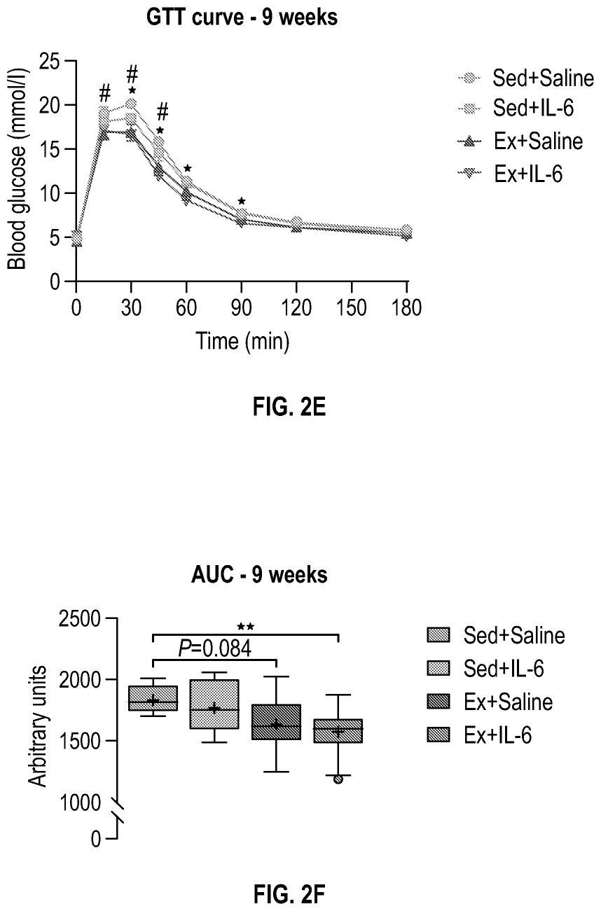

The Sed+Saline (FIG. 2B) group showed a decrease in glucose tolerance with increasing age, characterized by a higher glucose peak and elevated glucose levels until 60 min post injection of a glucose bolus (2 g glucose/kg body mass) compared to the baseline test, resulting in a higher area under the curve (AUC). These age-related changes in glucose tolerance are prevented in the rIL-6-treated sedentary group (FIG. 2B), and by endurance training with or without concomitant application of rIL-6 (FIGS. 2C and D), all of which resulted in significantly lower retest (9 weeks) GTT curves compared to the Sed+Saline group (FIG. 2E). Of note, while the retest AUC of the Ex+Saline group was not different from the other groups, the AUC of the Ex+IL-6 group was significantly smaller compared to the AUC of the Sed+Saline group (FIG. 2F) suggesting that besides the moderate effect in sedentary mice during aging, IL-6 may also act additively on exercise training.

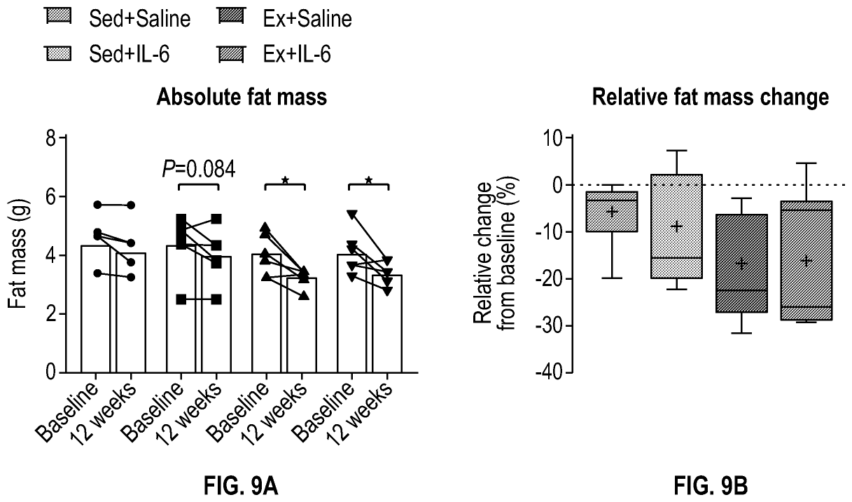

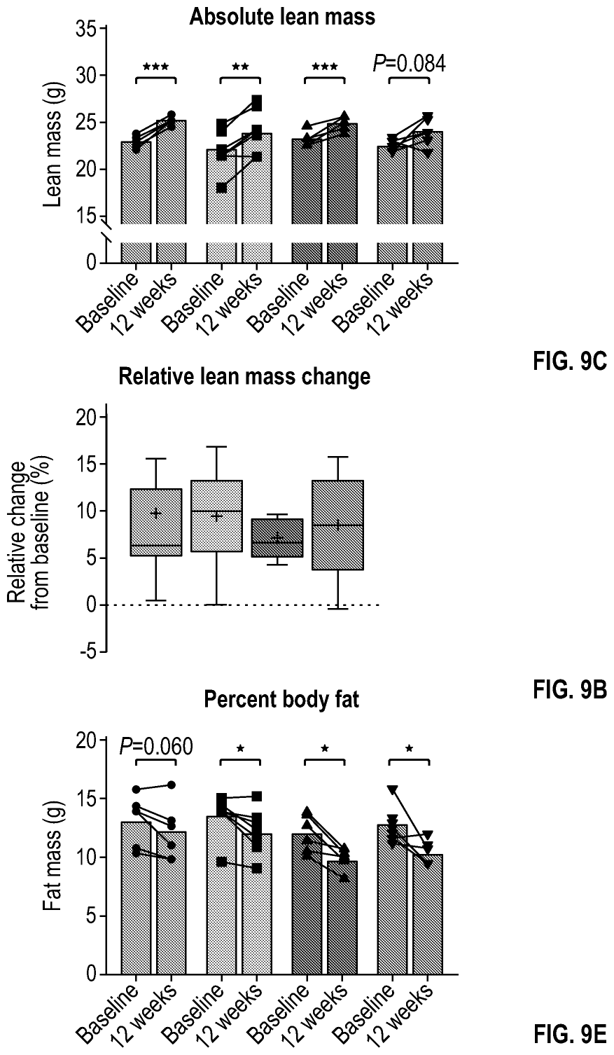

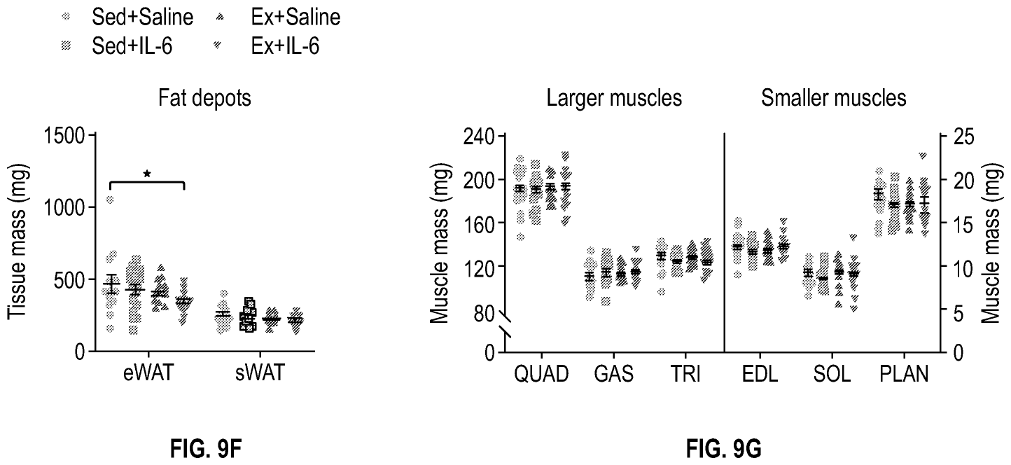

Exercise and/or rIL-6 treatment may affect glucose disposal by changing body composition (e.g. by increasing the amount of insulin-sensitive lean mass) and we therefore assessed changes in lean and fat mass in response to the interventions for a subgroup of mice by EchoMRI. After 12 weeks, fat mass was significantly reduced compared to baseline in the two exercise groups (FIGS. 9A and B), whereas lean mass increased in all groups (FIGS. 9C and D), except for the Ex+IL-6 group (P=0.084). This resulted in a significant overall reduction of body fat percentage after 12 weeks in the two exercise groups as well as in the Sed+IL-6, but not the Sed+Saline group (FIG. 9E). Interestingly, the Ex+IL-6 group had significantly less epididymal white adipose tissue (eWAT, a visceral fat depot in mice) compared to the Sed+Saline group, while no difference in subcutaneous white adipose tissue (sWAT) was detected at the end of the study (FIG. 9F). Furthermore, neither the exercise only nor the rIL-6 only group had a comparably low eWAT mass indicating that only the combination of extra rIL-6 with endurance training can effectively reduce eWAT tissue. In accordance with the lean body mass measurements, no differences in individual limb muscle masses were observed at the end of the study (FIG. 9G), even though the limb muscles of exercising mice were likely exposed to more mechanical and metabolic stress during the course of the intervention. Overall, this data imply that exercise and rIL-6 regulate glucose tolerance in old mice independent of large changes in lean mass, and therefore, by modulating other relevant processes such as insulin secretion and/or sensitivity.

Moderate-intensity low-volume endurance training prevents the age-related decline in \dot{V}O₂ₚₑₐₖ. Endurance exercise capacity and its physiological determinants, such as peak oxygen uptake (\dot{V}O₂ₚₑₐₖ), are strong independent predictors of mortality [50], and decrease with age [51]. We therefore checked whether \dot{V}O₂ₚₑₐₖ was affected by aging in our old mice and whether moderate endurance training and/or rIL-6 treatment modulated this parameter. Mice were subjected to a short and very intense ramp-sprint protocol (FIG. 3A) designed to determine \dot{V}O₂ₚₑₐₖ values at baseline and after 10 weeks of the intervention. Blood lactate levels of all four groups rose above 12 mmol/l in both the baseline and the retest, indicating that the animals were reaching their peak performance (FIG. 3B). \dot{V}O₂ₚₑₐₖ measurements revealed a pronounced age-related decline in the sedentary saline treated animals (16% decrease) that could not be prevented by rIL-6 treatment, as the Sed+IL-6 group displayed a similar decrease (15%) in \dot{V}O₂ₚₑₐₖ (FIG. 3C). Intriguingly, moderate exercise training appeared to be sufficient to preserve \dot{V}O₂ₚₑₐₖ completely with aging, indicating that these mice maintained their upper limit of aerobic metabolism within the observation period. There was, however, no detectable additive effect of extra rIL-6 on \dot{V}O₂ₚₑₐₖ, because the Ex+IL-6 group had no further increase in this parameter. Collectively, these results suggest that rIL-6 treatment did not have a strong direct effect on convective oxygen transport, i.e. on blood oxygenation, lung and/or heart function, as \dot{V}O₂ₚₑₐₖ is mainly determined by the ability of the cardiorespiratory system to deliver oxygen to exercising muscles [52]. Furthermore, neither exercise nor rIL-6 significantly altered maximal speed and distance in this exercise test (FIGS. 3D and 3E).

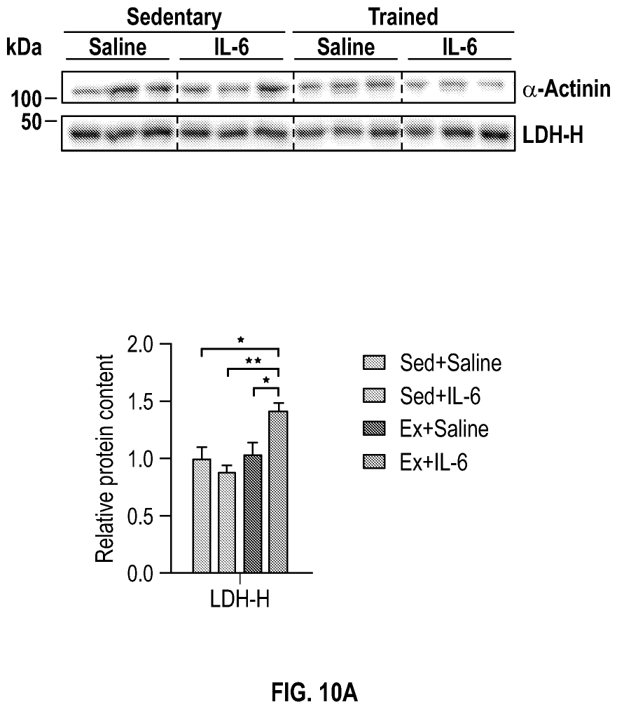

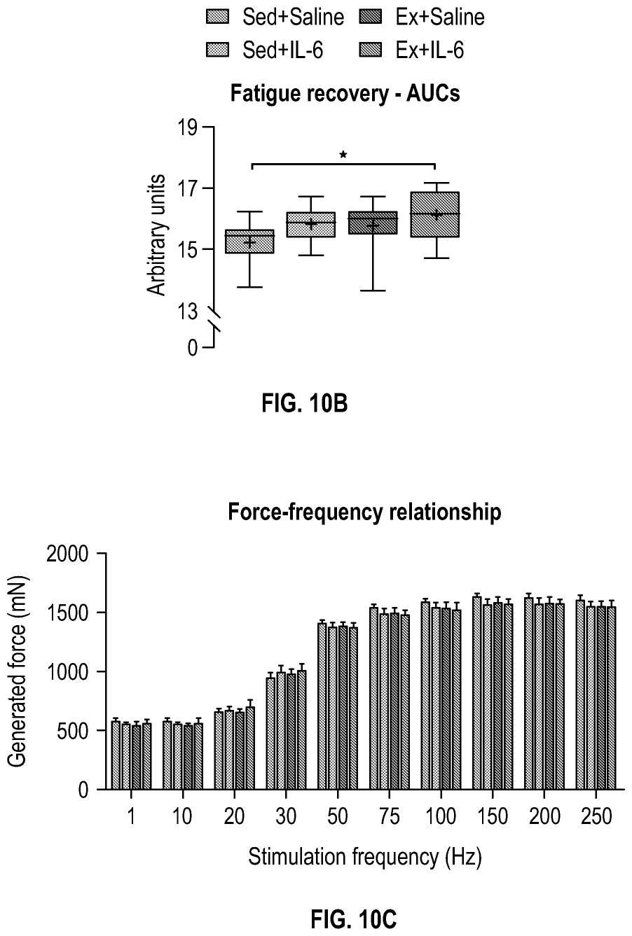

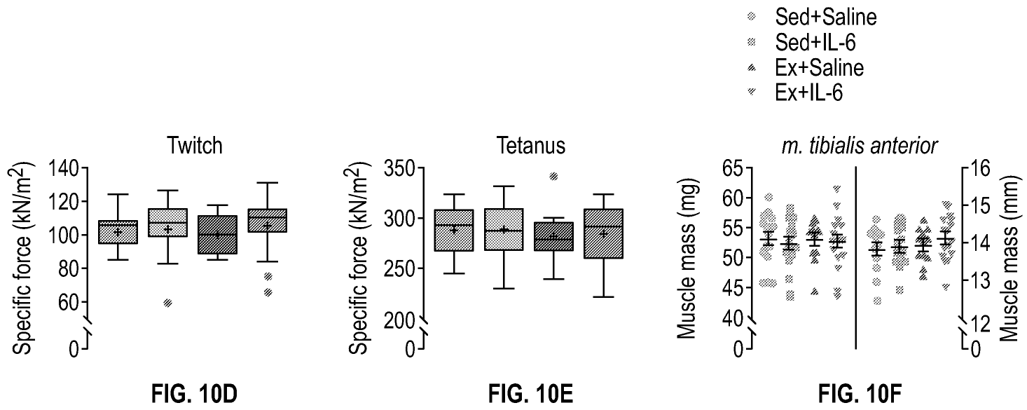

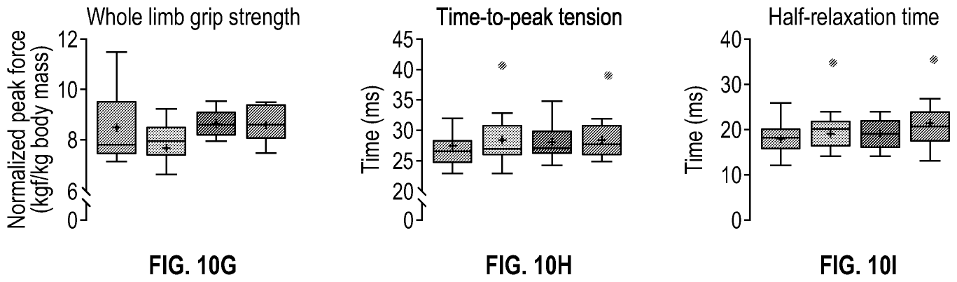

Elevated levels of IL-6 during endurance training bouts improves treadmill running capacity and muscle fatigue resistance in situ after 12 weeks of training. Results from the \dot{V}O₂ₚₑₐₖ test implied that there is no difference in the upper limit of exercise performance in short and very intense settings between saline and rIL-6 treated animals. However, besides the capacity of convective oxygen delivery, muscle intrinsic aspects, such as diffusive oxygen transport and metabolic processes, strongly determine submaximal endurance performance and fatigue resistance. Moreover, since mice are inherently good endurance runners and can usually cover large distances at intensities below \dot{V}O₂ₚₑₐₖ, running capacity and fatigue resistance may be more physiologically assessed by using a low intensity and long lasting protocol. We therefore challenged mice with a long duration incremental step protocol (FIG. 4A) at baseline and at the end of the intervention (12 weeks). In this test, the two sedentary groups showed no change in parameters measured to estimate endurance exercise capacity compared to baseline (FIG. 4B-F), indicating that despite the marked reduction in \dot{V}O₂ₚₑₐₖ, fatigue resistance at submaximal performance may be unaffected by aging within the observed period. Surprisingly, while having maintained the same level of \dot{V}O₂ₚₑₐₖ, performance of the two exercise groups was strongly diverging in this test. The mice that were exposed to higher levels of rIL-6 during exercise training sessions clearly improved their running capacity within the 12 weeks of training, as running distance covered (FIGS. 4B and C) and time to exhaustion (FIG. 4D) were both significantly longer compared to baseline. Moreover, these mice performed 33% more work (FIG. 4E) and reached a 12% greater peak power (FIG. 4F) in the retest at 12 weeks. In stark contrast, the Ex+Saline group, which underwent the same amount of endurance training as the Ex+IL-6 group, did not improve in any of the assessed parameters, and thus was more similar to the two sedentary control groups. In addition, measurements of blood lactate concentrations (a proxy for glycolytic stress and homeostatic disturbance) before (basal) and at the end (exhausted) of each test, indicated a “lactate threshold” decrease with aging, which was less pronounced when treated with rIL-6 or endurance trained, and even absent when rIL-6 and training were combined (FIG. 4G). While all four groups showed a clear increase in lactate concentrations upon exhaustion at baseline and at 12 weeks, the increase during the retest was significantly larger in the two sedentary groups as well as in the Ex+Saline group, but remained unchanged in the Ex+IL-6 group (FIG. 4H). Moreover, even though they performed 19% more work and had a 10% higher power output at exhaustion, the Ex+IL-6 mice accumulated significantly less lactate compared to the Sed+Saline mice in the retest (FIG. 4G). Western blot analysis for the lactate dehydrogenase heart subunit (LDH-H, transcribed from the Ldhb gene) that drives the conversion of lactate to pyruvate, revealed a higher abundance of this form in Ex+IL-6 mice compared to the other groups (FIG. 10A), suggesting enhanced capacity for lactate clearance in these animals.

To further characterize muscle intrinsic aspects, we assessed in situ fatigability and contractile properties of the m. tibialis anterior by sciatic nerve stimulation at the end of the study. The in situ set-up provides the advantage that the functional properties of the muscle can be assessed in its physiological environment, while motivational, central and cardiorespiratory components are excluded. In response to a four-minute fatigue protocol (consisting of intermittent 100 Hz tetanic stimulations), the Ex+IL-6 group showed the slowest drop in force production, leading to a clear separation of its fatigue curve from the curves of the other groups (FIG. 4I). Intriguingly, the resulting AUC (FIG. 4J) of the Ex+IL-6 group was not only significantly larger compared to the two sedentary groups, but also compared to the Ex+Saline group, which suggests that extra IL-6 during training sessions was required to increase fatigue resistance in response to the 12-weeks endurance training program in the m. tibialis anterior. Similarly, the Ex+IL-6 group showed the fastest recovery of all groups (FIG. 4I, FIG. 10B). Of note, the observed gain in muscular endurance did not appear to be at the expense of muscle force, as single twitch and tetanic forces in situ (FIG. 10C, force frequency curve, and FIGS. 10D and E, maximal twitch and tetanic force normalized to muscle cross-sectional area) as well as grip strength measures in vivo (FIG. 10G) were similar in all groups. Furthermore, contraction and relaxation velocities did not differ between groups (FIG. 10H, time-to-peak tension and FIG. 10I, half-relaxation time).

2. Administration of rIL-6 During Endurance Training Bouts Improves Gait and Motor Coordination in Old Aging Mice

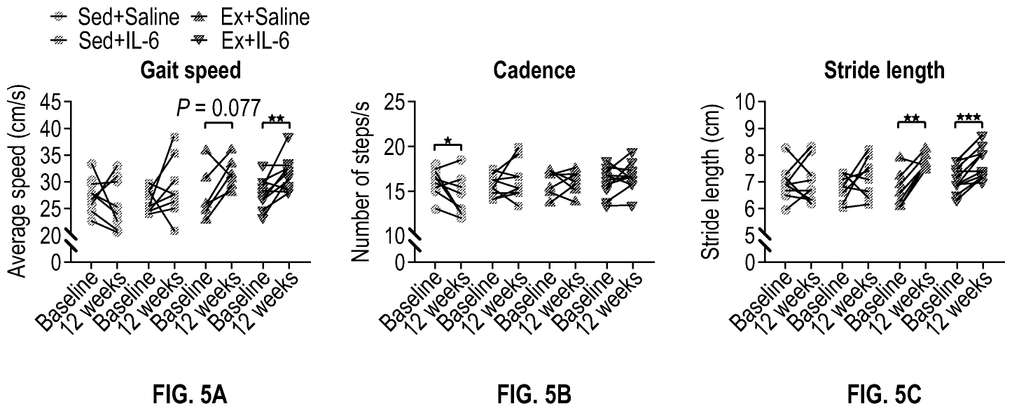

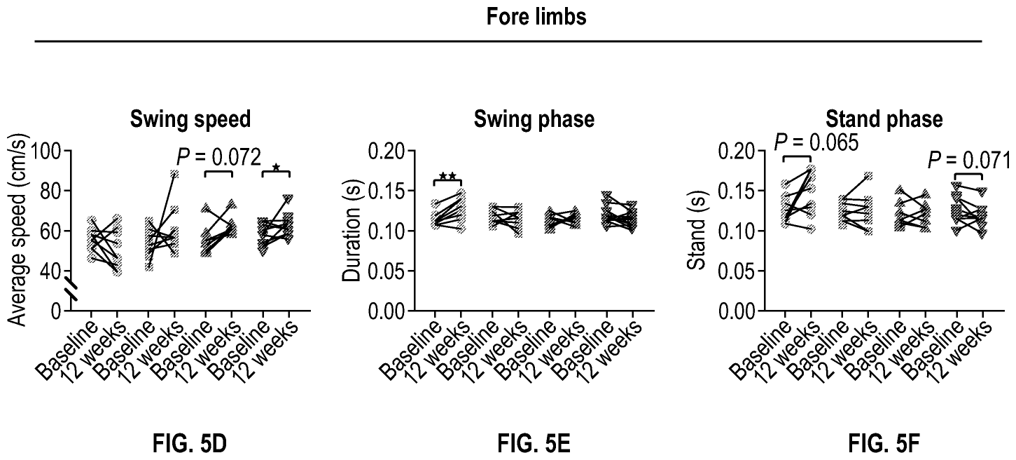

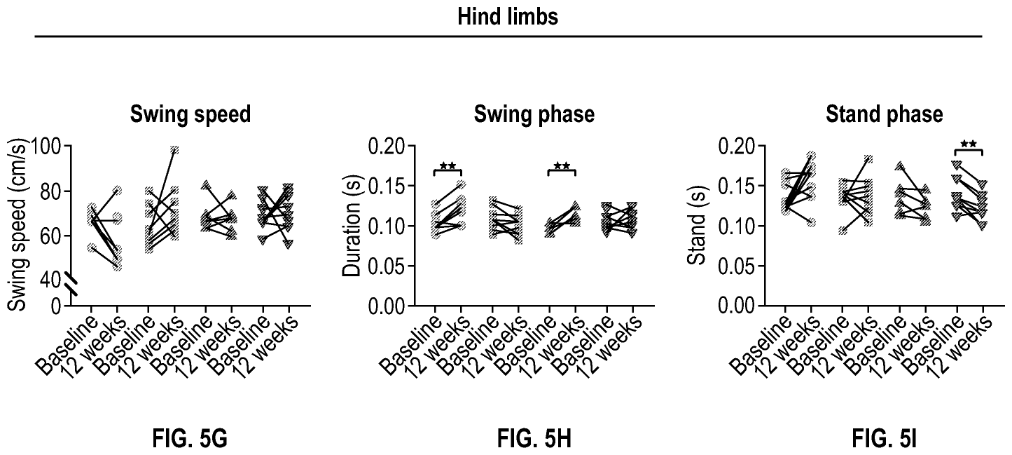

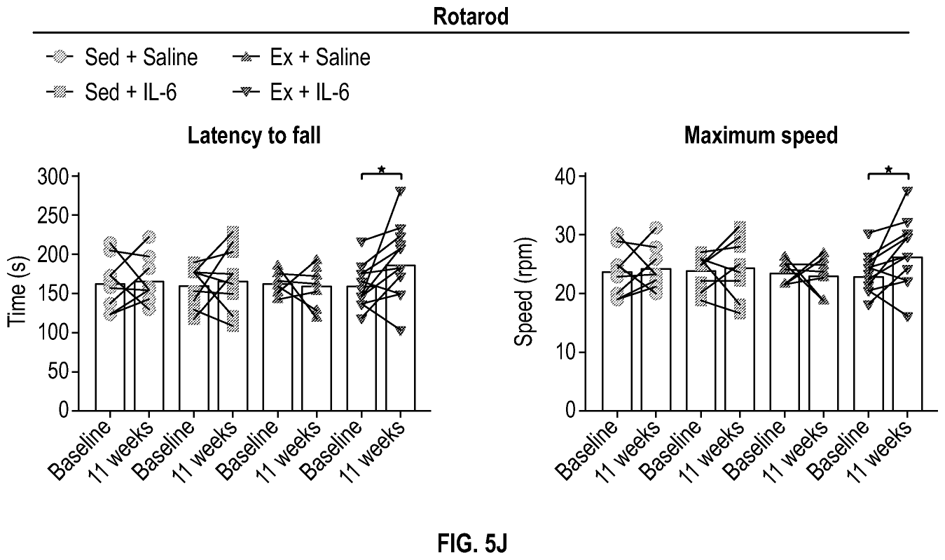

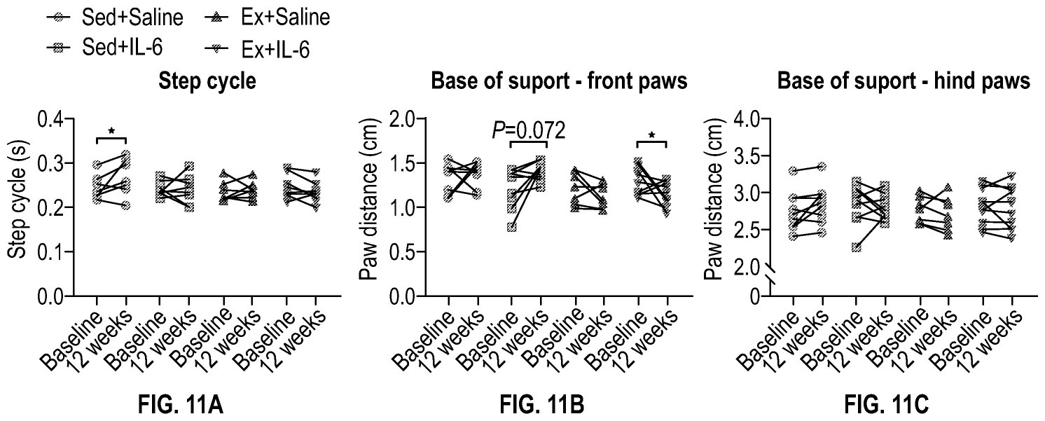

Walking speed is a strong independent predictor of life expectancy and unsteady gait and deficits in motor coordination largely contribute to the increased risk of falls and the ensuing devastating health consequences in elderly adults. We thus examined whether exercise and/or rIL-6 treatment modulated gait and motor coordination in aging mice by using the CatWalk XT voluntary gait analysis system and a Rotarod-based test. Compared to baseline, gait speed remained constant in both sedentary groups after 12 weeks (FIG. 5A), but the number of steps per second (cadence) appeared to decrease significantly in the Sed+Saline group (FIG. 5B). The decrease in cadence was mainly due to an increased duration of step cycle (sum of swing and stand phase, FIG. 11A), which was driven by an increase in the duration of swing phase of both hind- and forelimbs (FIGS. 5E and H). Therefore, aging led to slower limb movements in sedentary mice, which appeared to be prevented with rIL-6 treatment. Moreover, rIL-6 administration during exercise bouts, increased gait speed significantly after 12 weeks of training, whereas exercise alone could not do so (FIG. 5A). While cadence remained unchanged (FIG. 5B), overall (i.e. hind- and forelimbs combined) stride length increased significantly in both exercise groups (FIG. 5C), suggesting that exercise induced an increase in step size, which was, however, not sufficient to improve gait speed. The additional increase in swing speed of forelimbs (FIG. 5D) and decrease in stand phase of hindlimbs (FIG. 5I) achieved by Ex+IL-6 mice, together with the increase in hindlimb swing phase in the Ex+Saline group (FIG. 5H), might explain the observed discrepancy in gait speed. In addition, Ex+IL-6 mice reduced the base of support of their forepaws, which is indicative of a more confident and secure gait (FIG. 11B). In accordance with the observed changes in voluntary gait, the Ex+IL-6 group was the only group that improved Rotarod performance within the treatment period, whereas performance of all other groups remained on baseline levels (FIG. 5J).

3. Exercised Mice Treated with rIL-6 Show Increased Expression of Mitochondrial Complex I Components and PDK4

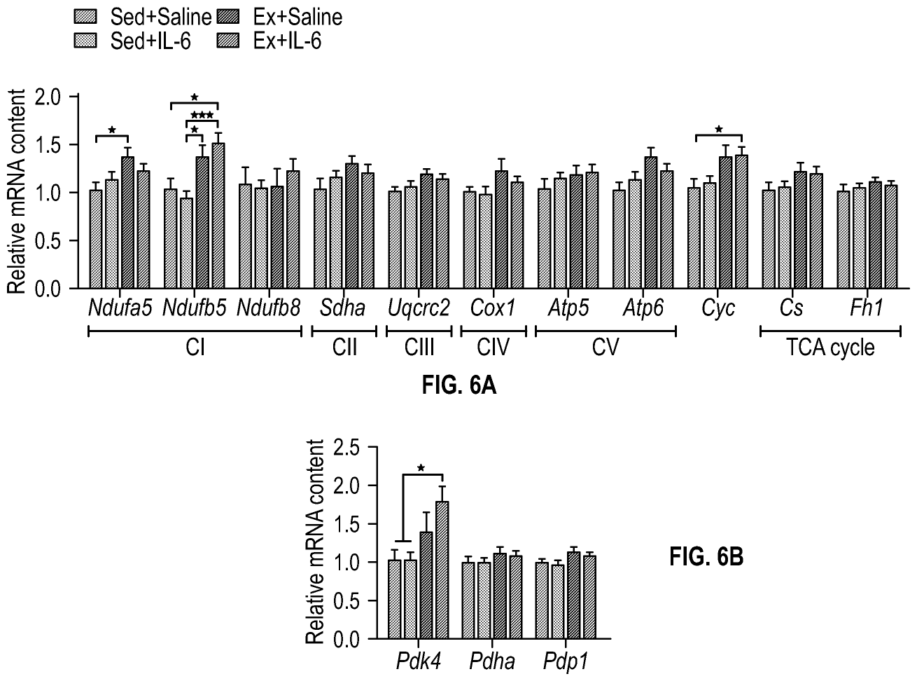

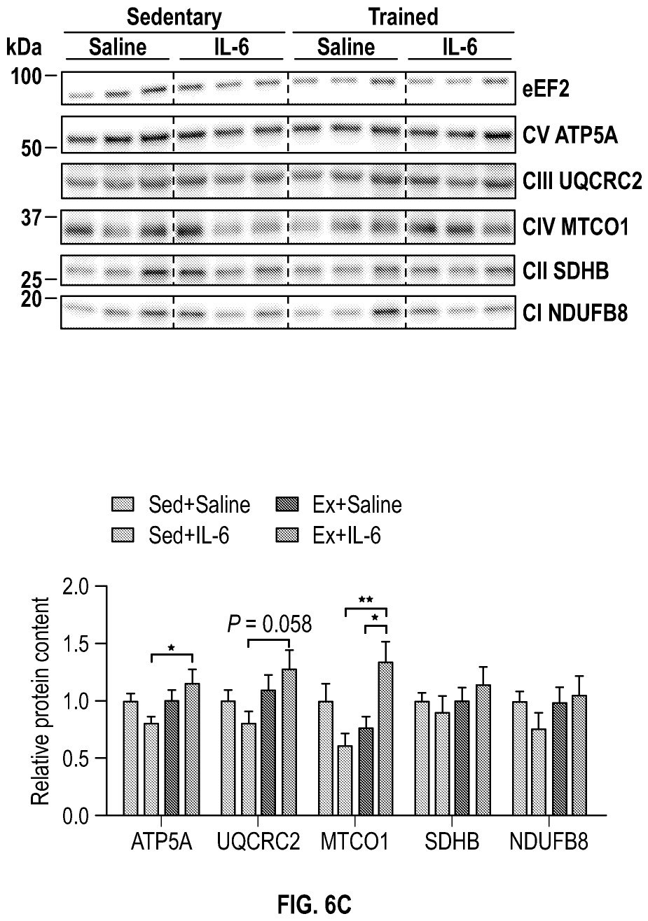

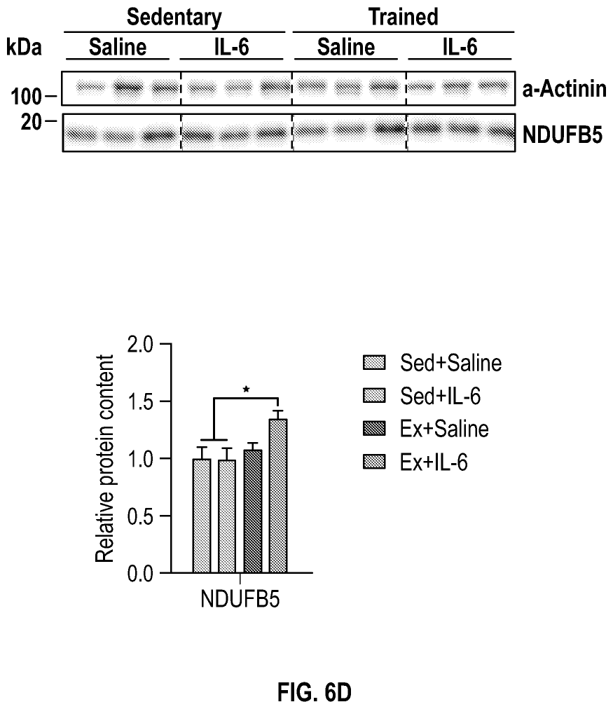

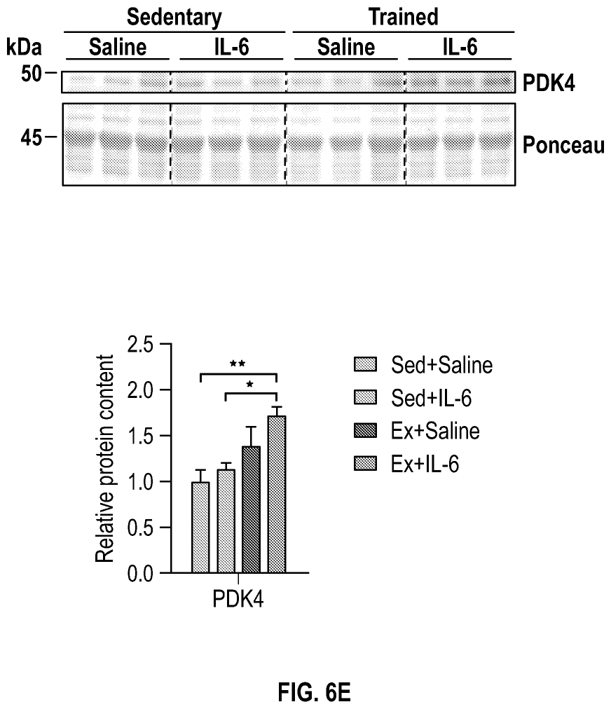

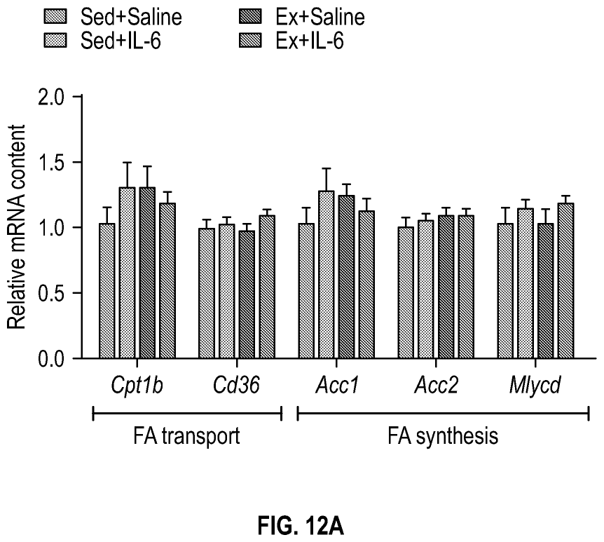

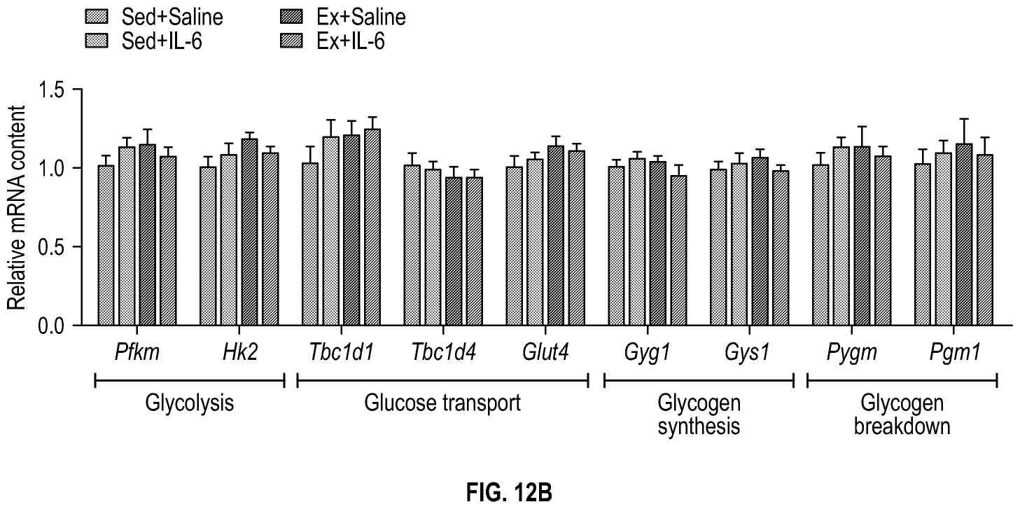

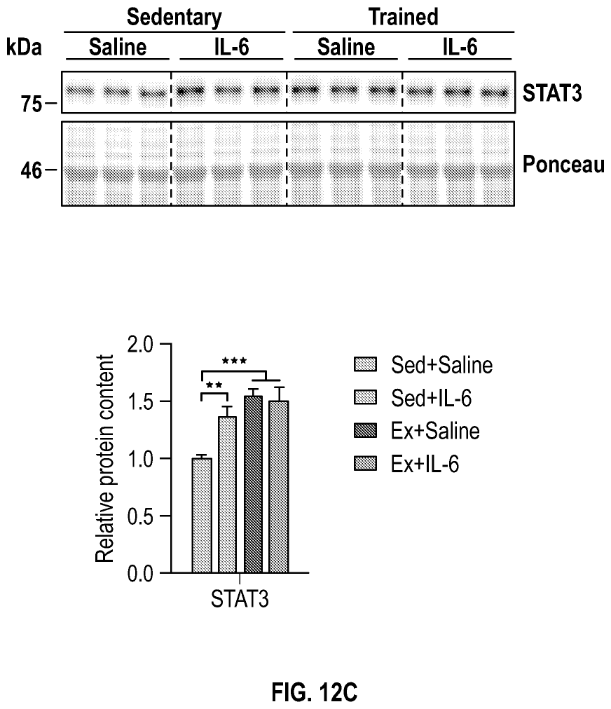

The fact that only Ex+IL-6 mice improved fatigue resistance in situ together with the observation that rIL-6 increased running performance in vivo without affecting \dot{V}O₂ₚₑₐₖ (i.e. convective oxygen transport) suggests that IL-6 potentiates skeletal muscle intrinsic adaptations to training. To get more insights on molecular changes, we extracted RNA from the m. quadriceps femoris (a muscle heavily used during treadmill running and displaying mixed fiber type composition) and performed quantitative polymerase chain reaction (qPCR) of metabolic genes. This analysis revealed no strong transcriptional differences between groups in key components of the electron transport chain (ETC) except for complex I (FIG. 6A). Moreover, genes encoding for proteins involved in fatty acid transport and synthesis as well as components involved in glucose metabolism and storage, remained unaffected by either intervention (FIGS. 12A and B). In contrast, pyruvate dehydrogenase kinase 4 (Pdk4), a gene encoding for a kinase that inactivates pyruvate dehydrogenase (PDH), which in turn is a positive regulator of carbohydrate-derived energy substrate utilization in mitochondria [54, 55], was upregulated in muscles of Ex+IL-6 mice (FIG. 6B), while the PDH component subunit E1 alpha (Pdha1) and pyruvate dehydrogenase phosphatase 1 (Pdp1, encoding a PDH activating phosphatase) showed similar expression in all groups.

An acute exercise bout and/or rIL-6 injection may induce transcriptional changes that turn back to baseline within 48 h but still affect protein abundance. Therefore, to study whether the transcriptional status is reflecting protein levels at the time of analysis, we performed western blots for OXPHOS proteins and PDK4 with tissue from the same muscles. Ex+IL-6 mice showed higher complex IV protein abundance in the OXPHOS blot, however, only significantly compared to the Sed+IL-6 and Ex+Saline group, as these mice displayed a slight decrease compared to the Sed+Saline mice (FIG. 6C). An additional western blot for the complex I component NDUFB5, which showed clearest difference in gene expression, revealed a slight upregulation in the Ex+IL-6 group (FIG. 6D). Also in accordance with gene expression, PDK4 protein levels were clearly elevated in IL-6 treated exercised mice (FIG. 6E). The increase in OXPHOS proteins and enhanced levels of PDK4 in skeletal muscle is indicative of a boosted oxidative capacity and fatty acid-derived substrate flux, which may provide performance advantage during prolonged exercise at the submaximal level. The higher PDK4 levels may also ameliorate glycogen replenishing following exercise. Of note, total protein levels of the downstream effector of IL-6 signaling, signal transducer and activator of transcription 3 (STAT3), were increased in skeletal muscle by both, rIL-6 treatment and endurance exercise training (FIG. 12C).

### D. Discussion

The age-associated decline in skeletal muscle function is one of the main drivers of loss-of-independence, admission to nursing homes, increased risk for chronic diseases, morbidity and mortality in the elderly. The only efficacious method to prevent and mitigate sarcopenia, frailty and other pathologies associated with this functional decline are exercise-based, both using resistance training to address loss in muscle mass and strength, and endurance training to improve cardiovascular function, fatigue, and frailty. Unfortunately, training interventions are notoriously difficult to implement and adhere to, in the general population, but, due to pre-existing frailty, impaired gait as well as reduced balance and motor coordination, co-morbidities and other events, in particular in the older individuals [4]. We therefore have now assessed how a low-volume, moderate-intensity endurance training could be combined with the application of rIL-6 to achieve synergy in improving the functional capacity of skeletal muscle in old mice.

Notably, even when only performed at old age, and despite the low volume, endurance training conferred potent beneficial effects on various parameters, most of which could not be recapitulated by the pulsatile administration of rIL-6 as performed in this study. Importantly however, when combined with training, rIL-6 powerfully enhanced a broad spectrum of biological programs that are impaired in aging. Indeed, following 12 weeks of training, Ex+IL-6 mice displayed higher endurance performance and prolonged contractions at higher relative force in situ, which excelled the effects of training. The combined treatment thereby most efficiently prevented the age-associated decline in ramp sprint capacity, improved fatigability, and accelerated muscle recovery in old mice. Second, we observed an age-related decrease in glucose tolerance, which was prevented with moderate-intensity low-volume endurance training and more moderately ameliorated with rIL-6 treatment in sedentary mice. However, only the combination of rIL-6 and training significantly lowered AUC in the post-intervention GTTs, hence even surpassing the already potent effect of training. Glucose tolerance is indicative of metabolic flexibly and metabolic dysfunction, manifested as impaired glucose tolerance, is a hallmark of the age-related metabolic syndrome. Often, insulin sensitivity and glucose tolerance are closely linked to body composition. In our cohorts, both training groups exhibited a reduction in fat mass, but only in the combination group (Ex+IL6), a significant decrease in the mass of eWAT was observed. In contrast to sWAT, excessive eWAT has been linked to pro-inflammatory events and the development of insulin resistance and type 2 diabetes [56]. This potential therapeutic effect of rIL-6 in combination with exercise on skeletal muscle-autonomous as well as systemic functions is underlined by the third important outcome, the ameliorated gait and motor coordination, none of which could be improved to a similar extent in mice trained without additional rIL-6. Even though increased muscular endurance is important for motor coordination, neuronal changes (e.g. vestibular and proprioceptive input, nerve conduction velocity or neuromuscular junction functionality) and motor planning are additional crucial components that could potentially be affected by IL-6. Intriguingly, rIL-6 treatment has been shown to be protective in chemotherapy- and diabetes-induced neuropathies [39-42], alluding to a direct effect of rIL-6 on neuronal integrity and potential function.

Similar to the age-related reduction in maximum aerobic capacity observed in humans [51], our mice experienced a drop in \dot{V}O₂ₚₑₐₖ during the study. A moderate-intensity low-volume endurance training applied over a three-month period was, however, successful in preserving \dot{V}O₂ₚₑₐₖ completely, demonstrating that a relatively small dose of exercise training is sufficient to prevent decrements in maximum performance at older age. In contrast, pulsatile rIL-6 treatment mimicking the transient increase of this cytokine in response to exercise could not mitigate the decrease in \dot{V}O₂ₚₑₐₖ in sedentary mice. Furthermore, extra rIL-6 during exercise sessions was not able to increase \dot{V}O₂ₚₑₐₖ in response to training. These findings are in accordance with two recent studies on human participants, revealing no impairments in \dot{V}O₂ₚₑₐₖ increase in response to 12 weeks of endurance training in obese or type two diabetic patients when IL-6 signaling is blocked with the IL-6 receptor antibody tocilizumab [32, 57].

Similarly, studies comparing wild type mice to global IL-6 knockouts did not observe differences in baseline \dot{V}O₂ₚₑₐₖ or differential effects of training on this parameter [58, 59]. Despite the equal values of \dot{V}O₂ₚₑₐₖ treadmill-running capacity (assessed with a long-duration incremental step protocol) increased remarkably in animals that were exposed to higher levels of IL-6 during single endurance training bouts, whereas endurance training alone was not sufficient to elevate any of the measured parameters. Therefore, the two exercise groups may differ in the proportion of \dot{V}O₂ₚₑₐₖ (i.e. the rate of “fractional utilization”) they can sustain during prolonged exercise. It is well known from studies in humans that individuals with a similar \dot{V}O₂ₚₑₐₖ can differ largely in performance at the submaximal level and that an increase in \dot{V}O₂ₚₑₐₖ in response to training does not always translate into enhanced performance [60]. Inversely, muscle intrinsic improvements such as, mitochondrial biogenesis or upregulated oxidative enzymes, may occur without changes in \dot{V}O₂ₚₑₐₖ [59, 60]. The fact that Ex+IL-6 mice had less lactate accumulation at exhaustion despite the higher power output supports the notion that adaptations within skeletal muscle are responsible for the improved performance, as higher lactate thresholds are often associated with training-induced increases in skeletal muscle oxidative capacity and optimized production, disposal and clearance of lactate [61]. For example, muscle and blood lactate concentrations start to increase around 60%-65% of \dot{V}O₂ₚₑₐₖ in untrained individuals but at 80%-90% of \dot{V}O₂ₚₑₐₖ in highly trained endurance athletes [60].

The systemic effects of rIL-6 on neuronal, adipose and other tissues will be investigated in future projects. In skeletal muscle, despite the pulsatile administration, rIL-6 elevated basal STAT3 levels similar to what was observed in endurance-trained old mice in our study. STAT3 signaling is involved in mediating some of the effects of exercise on skeletal muscle metabolism and performance. For example, STAT3 is transiently activated in muscle fibers by resistance exercise in young and aged muscle, which is preserved following training suggesting a potential role for STAT3 signaling in the physiological adaptation to resistance training. More recently, Knudsen et al. showed in young mice that endurance exercise-induced STAT3 activation in skeletal muscle fibers is necessary for the beneficial effects of training on exercise performance and glucose homeostasis, which according to the authors may be mediated by the cytokine IL-13. While STAT3 exerts many effects in different cells, recent evidence points towards a direct regulatory link between this transcription factor and metabolic genes. For example, in human prostate tumors, low Stat3 expression was associated with low Pdk4 expression, and subsequent analyses of several existing and newly acquired datasets from chromatin immunoprecipitation DNA-sequencing (ChIP-Seq) showed binding of STAT3 to the promoter region of Pdk4 [66]. In addition, ChIP assays on control and shSTAT3 cells with or without prior IL-6 stimulation showed highest STAT3 levels and Pdk4 promoter region binding with IL-6 stimulation and a reduction with Stat3 knockdown [66]. In line with these reports, exercise combined with rIL-6 levels upregulated resting levels of PDK4 gene and protein expression in skeletal muscle in our study. PDK4 is an inhibitor of PDH, which in turn is a positive regulator of carbohydrate-derived energy substrate utilization in mitochondria. In human skeletal muscle, PDH activity increases during the initial 120 min of an exercise bout to subsequently decrease toward the resting level as the exercise duration proceeds [67-69], which is in accordance with an increasing proportion of fat oxidation during prolonged muscular activity. Higher PDK4 levels may thus allow a rapid or more efficient upregulation of fatty acid oxidation during exercise and thereby providing performance advantage by sparing glycogen. It has previously been shown that a single injection of rIL-6 decreases skeletal muscle PDH activity in mice in the fed state [70], and that skeletal muscle PDH activity is higher in muscle-specific knockouts compared to controls at rest and at 60 min of exercise [71]. Concomitant with elevated PDK4, the combination of training and rIL-6 increased the transcript and protein levels of several components of the mitochondrial respiratory chain, and those of LDH-B in our study. Collectively, this metabolic remodeling could favor more efficient oxidation of lipids and lactate, and thereby contribute to the higher endurance and fatigue resistance in this group.

Many therapeutic approaches aim at neutralizing the pro-inflammatory effects of IL-6 and hence, application of exogenous rIL-6 could theoretically be regarded as an unsafe approach. To mitigate potential deleterious effects of persistently elevated IL-6 as observed in many chronic diseases, we therefore aimed at a pulsatile, low-dose application replicating the regulation of systemic IL-6 as a myokine during and after acute exercise bouts. Since IL-6 levels return to baseline within ˜2 h after injection [49], IL-6 was indeed not chronically elevated in terminal plasma measurements in our mice. Second, the absence of any specific change in other pro-inflammatory cytokines, e.g. TNF-α, IL-1β or INF-γ, in the rIL-6-treated groups compared to the levels in the control cohort indicate that the application of rIL-6 did not induce a pro-inflammatory environment. This conclusion is supported by the absence of any pyrogenic activity, modulation of spontaneous locomotion, body mass or food intake by rIL-6. Furthermore, inconspicuous ALT and AST levels in the rIL-6 groups indicate normal liver health, and the reduction in plasma LDH by rIL-6 and/or training could imply an even reduced cellular damage in these mice. Collectively, these data demonstrate that a pulsatile, low-dose administration of rIL-6, with or without concomitant endurance training, is safe, well tolerated, and lacks any discernable adverse effects in old mice.

IL-6 is thought to improve acute exercise adaptation and performance by orchestrating the supply of energy substrates to working muscles and by facilitating their uptake and catabolism [27-33]. In accordance, mice lacking IL-6 globally [58, 59, 72-75] or muscle specifically have consistently been shown to display lower (acute) exercise tolerance and a blunted induction of metabolic enzymes and nutrient uptake into skeletal muscle. Our observation that rIL-6 administration during exercise sessions leads to increased fatigue resistance in response to long-term training now suggests that IL-6 also regulates training adaptation. Importantly, potential acute performance enhancing effects of rIL-6 injections can likely be excluded in our study, since all tests were performed under basal conditions i.e., at least 48 h after the last injection. Previous studies have revealed that IL-6 is not necessary for mitochondrial biogenesis or improvements in cardio-respiratory fitness in abdominally obese adult humans [32]. In contrast, IL-6 might favor the upregulation of cyclooxygenase and citrate synthase activity following training and, in abdominally obese humans, may be required for the reduction of visceral and cardiac adipose tissue mass as well as the gain in left ventricle mass in response to 12 weeks of endurance training [32, 77]. However, even though IL-6 is the most extensively studied myokine in both animals and humans, most studies of rIL-6 administration focused only on acute effects and studies investigating the outcome of repeatedly enhancing systemic IL-6 levels and/or signaling in skeletal muscle are lacking. Therefore, to the best of our knowledge, the present study is the first to suggest a function of IL-6 in mediating training induced performance improvements.

### E. Conclusions

Our results demonstrate that the application of pulsatile, low-dose rIL-6 can potentiate the beneficial effects of a low-volume endurance training on muscle intrinsic and systemic parameters in old mice. Of note, no detectable adverse effects of this treatment were observed, implying that such an intervention is safe and well tolerated. Even though future studies will aim at a careful investigation of sarcopenia, the present data clearly demonstrate improvements in glucose tolerance, endurance performance, fatigue resistance and muscle recovery, gait and motor coordination. Thus, if this intervention can be translated to elderly individuals, rIL-6 could not only facilitate training interventions, and hence increase adherence and compliance, but also directly result in a massive improvement of quality of life by affecting gait and motor coordination. Importantly, fatigability, a key hallmark of human frailty [80], could likewise be significantly mitigated. Collectively, these improvements could alleviate insecurity, avoidance of physical activity and exercise, falls and the ensuing fractures and hospitalizations, and the loss-of-independence as well as admission to nursing homes might be delayed. Clinical trials and safety studies with rIL-6 have already been performed in non-geriatric individuals. Therefore, following a careful evaluation of safety, tolerability and adverse effects in the elderly, the use of combined rIL-6-training interventions in the prevention and treatment of age-associated functional decline could be initiated in a relatively short amount of time.

### F. REFERENCES

1. Lopez-Otin C, Blasco M A, Partridge L, Serrano M, Kroemer G. The hallmarks of aging. Cell. 2013; 153:1194-217. doi:10.1016/j.cell.2013.05.039
2. Booth F W, Roberts C K, Laye M J. Lack of exercise is a major cause of chronic diseases. Compr Physiol. 2012; 2:1143-211. doi: 10.1002/cphy.c110025
3. Bouchard C, Blair S N, Katzmarzyk P T. Less Sitting, More Physical Activity, or Higher Fitness? Mayo Clin Proc. 2015; 90:1533-40. doi:10.1016/j.mayocp.2015.08.005
4. Billot M, Calvani R, Urtamo A, Sanchez-Sanchez J L, Ciccolari-Micaldi C, Chang M, et al. Preserving Mobility in Older Adults with Physical Frailty and Sarcopenia: Opportunities, Challenges, and Recommendations for Physical Activity Interventions. Clin Interv Aging. 2020; 15:1675-90. doi:10.2147/CIA.S253535
5. Oikawa S Y, Holloway™, Phillips S M. The Impact of Step Reduction on Muscle Health in Aging: Protein and Exercise as Countermeasures. Front Nutr. 2019; 6:75. doi: 10.3389/fnut.2019.00075
6. Pinedo-Villanueva R, Westbury L D, Syddall H E, Sanchez-Santos M T, Dennison E M, Robinson S M, et al. Health Care Costs Associated With Muscle Weakness: A UK Population-Based Estimate. Calcif Tissue Int. 2019; 104:137-44. doi:10.1007/s00223-018-0478-1
7. Sousa A S, Guerra R S, Fonseca I, Pichel F, Ferreira S, Amaral T F. Financial impact of sarcopenia on hospitalization costs. Eur J Clin Nutr. 2016; 70:1046-51. doi:10.1038/ejcn.2016.73
8. Harper S. Economic and social implications of aging societies. Science. 2014; 346:587-91. doi:10.1126/science.1254405
9. Harridge S D, Lazarus N R. Physical Activity, Aging, and Physiological Function. Physiology (Bethesda). 2017; 32:152-61. doi:10.1152/physiol.00029.2016
10. Pedersen B K, Saltin B. Exercise as medicine-evidence for prescribing exercise as therapy in 26 different chronic diseases. Scand J Med Sci Sports. 2015; 25 Suppl 3:1-72. doi:10.1111/sms.12581
11. McKendry J, Breen L, Shad B J, Greig C A. Muscle morphology and performance in master athletes: A systematic review and meta-analyses. Ageing Res Rev. 2018; 45:62-82. doi:10.1016/j.arr.2018.04.007
12. Garatachea N, Pareja-Galeano H, Sanchis-Gomar F, Santos-Lozano A, Fiuza-Luces C, Moran M, et al. Exercise attenuates the major hallmarks of aging. Rejuvenation Res. 2015; 18:57-89. doi:10.1089/rej.2014.1623
13. Kumar V, Selby A, Rankin D, Patel R, Atherton P, Hildebrandt W, et al. Age-related differences in the dose-response relationship of muscle protein synthesis to resistance exercise in young and old men. J Physiol. 2009; 587:211-7. doi:10.1113/jphysiol.2008.164483
14. Fry C S, Drummond M J, Glynn E L, Dickinson J M, Gundermann D M, Timmerman K L, et al. Aging impairs contraction-induced human skeletal muscle mTORC1 signaling and protein synthesis. Skelet Muscle. 2011; 1:11. doi:10.1186/2044-5040-1-11
15. West D W D, Marcotte G R, Chason C M, Juo N, Baehr L M, Bodine S C, et al. Normal Ribosomal Biogenesis but Shortened Protein Synthetic Response to Acute Eccentric Resistance Exercise in Old Skeletal Muscle. Front Physiol. 2018; 9:1915. doi:10.3389/fphys.2018.01915
16. Bickel C S, Cross J M, Bamman M M. Exercise dosing to retain resistance training adaptations in young and older adults. Med Sci Sports Exerc. 2011; 43:1177-87. doi:10.1249/MSS.0b013e318207c15d
17. Hughes D C, Marcotte G R, Baehr L M, West D W D, Marshall A G, Ebert S M, et al. Alterations in the muscle force transfer apparatus in aged rats during unloading and reloading: impact of microRNA-31. J Physiol. 2018; 596:2883-900. doi: 10.1113/JP275833
18. Brooks S V, Faulkner J A. The magnitude of the initial injury induced by stretches of maximally activated muscle fibres of mice and rats increases in old age. J Physiol. 1996; 497 (Pt 2):573-80. doi:10.1113/jphysiol.1996.sp021790
19. Joanisse S, Nederveen J P, Snijders T, Mckay B R, Parise G. Skeletal Muscle Regeneration, Repair and Remodelling in Aging: The Importance of Muscle Stem Cells and Vascularization. Gerontology. 2017; 63:91-100. doi: 10.1159/000450922
20. Grounds M D. Age-associated changes in the response of skeletal muscle cells to exercise and regeneration. Ann N Y Acad Sci. 1998; 854:78-91. doi: 10.1111/j. 1749-6632.1998.tb09894.x
21. Hung W W, Ross J S, Boockvar K S, Siu A L. Recent trends in chronic disease, impairment and disability among older adults in the United States. BMC Geriatr. 2011; 11:47. doi: 10.1186/1471-2318-11-47
22. Leuchtmann A B, Handschin C. Pharmacological targeting of age-related changes in skeletal muscle tissue. Pharmacol Res. 2019; 104191. doi:10.1016/j.phrs.2019.02.030
23. Pedersen B K, Febbraio M A. Muscles, exercise and obesity: skeletal muscle as a secretory organ. Nat Rev Endocrinol. 2012; 8:457-65. doi: 10.1038/nrendo.2012.49
24. Schnyder S, Handschin C. Skeletal muscle as an endocrine organ: PGC-1alpha, myokines and exercise. Bone. 2015; 80:115-25. doi:10.1016/j.bone.2015.02.008
25. Pedersen B K, Febbraio M A. Muscle as an endocrine organ: focus on muscle-derived interleukin-6. Physiol Rev. 2008; 88:1379-406. doi:10.1152/physrev.90100.2007
26. Steensberg A, Febbraio M A, Osada T, Schjerling P, van Hall G, Saltin B, et al. Interleukin-6 production in contracting human skeletal muscle is influenced by pre-exercise muscle glycogen content. J Physiol. 2001; 537:633-9. doi: 10.1111/j.1469-7793.2001.00633.x
27. Febbraio M A, Hiscock N, Sacchetti M, Fischer C P, Pedersen B K. Interleukin-6 is a novel factor mediating glucose homeostasis during skeletal muscle contraction. Diabetes. 2004; 53:1643-8.
28 Stouthard J M, Romijn J A, Van der Poll T, Endert E, Klein S, Bakker P J, et al. Endocrinologic and metabolic effects of interleukin-6 in humans. Am J Physiol. 1995; 268:E813-9. doi: 10.1152/ajpendo.1995.268.5.E813
29 van Hall G, Steensberg A, Sacchetti M, Fischer C, Keller C, Schjerling P, et al. Interleukin-6 stimulates lipolysis and fat oxidation in humans. J Clin Endocrinol Metab. 2003; 88:3005-10. doi:10.1210/jc.2002-021687
30. Wolsk E, Mygind H, Grondahl T S, Pedersen B K, van Hall G. IL-6 selectively stimulates fat metabolism in human skeletal muscle. Am J Physiol Endocrinol Metab. 2010; 299:E832-40. doi: 10.1152/ajpendo.00328.2010
31. Bruce C R, Dyck D J. Cytokine regulation of skeletal muscle fatty acid metabolism: effect of interleukin-6 and tumor necrosis factor-alpha. Am J Physiol Endocrinol Metab. 2004; 287:E616-21. doi: 10.1152/ajpendo.00150.2004
32. Wedell-Neergaard A S, Lang Lehrskov L, Christensen R H, Legaard G E, Dorph E, Larsen M K, et al. Exercise-Induced Changes in Visceral Adipose Tissue Mass Are Regulated by IL-6 Signaling: A Randomized Controlled Trial. Cell Metab. 2019; 29:844-55 e3. doi:10.1016/j.cmet.2018.12.007
33. Chowdhury S, Schulz L C, Palmisano B, Singh P, Berger J M, Yadav V K, et al. Muscle derived interleukin-6 increases exercise capacity by signaling in osteoblasts. J Clin Invest. 2020; doi: 10.1172/JCI133572
34. Munoz-Canoves P, Scheele C, Pedersen B K, Serrano A L. Interleukin-6 myokine signaling in skeletal muscle: a double-edged sword? FEBS J. 2013; 280:4131-48. doi:10.1111/febs.12338
35. Petersen A M, Pedersen B K. The anti-inflammatory effect of exercise. J Appl Physiol (1985). 2005; 98:1154-62. doi:10.1152/japplphysiol.00164.2004
36. Benatti F B, Pedersen B K. Exercise as an anti-inflammatory therapy for rheumatic diseases-myokine regulation. Nat Rev Rheumatol. 2015; 11:86-97. doi:10.1038/nrrheum.2014.193
37. Starkie R, Ostrowski S R, Jauffred S, Febbraio M, Pedersen B K. Exercise and IL-6 infusion inhibit endotoxin-induced TNF-alpha production in humans. FASEB J. 2003; 17:884-6. doi:10.1096/fj.02-0670fje
38. Ostrowski K, Rohde T, Asp S, Schjerling P, Pedersen B K. Pro- and anti-inflammatory cytokine balance in strenuous exercise in humans. J Physiol. 1999; 515 (Pt 1):287-91.
39. Callizot N, Andriambeloson E, Glass J, Revel M, Ferro P, Cirillo R, et al. Interleukin-6 protects against paclitaxel, cisplatin and vincristine-induced neuropathies without impairing chemotherapeutic activity. Cancer Chemother Pharmacol. 2008; 62:995-1007. doi: 10.1007/s00280-008-0689-7
40. Andriambeloson E, Baillet C, Vitte P A, Garotta G, Dreano M, Callizot N. Interleukin-6 attenuates the development of experimental diabetes-related neuropathy. Neuropathology. 2006; 26:32-42.
41. Cameron N E, Cotter M A. The neurocytokine, interleukin-6, corrects nerve dysfunction in experimental diabetes. Exp Neurol. 2007; 207:23-9. doi:10.1016/j.expneurol.2007.05.009
42. Cotter M A, Gibson T M, Nangle M R, Cameron N E. Effects of interleukin-6 treatment on neurovascular function, nerve perfusion and vascular endothelium in diabetic rats. Diabetes Obes Metab. 2010; 12:689-99. doi: 10.1111/j.1463-1326.2010.01221.x
43. Delezie J, Weihrauch M, Maier G, Tejero R, Ham D J, Gill J F, et al. BDNF is a mediator of glycolytic fiber-type specification in mouse skeletal muscle. Proc Natl Acad Sci USA. 2019; 116:16111-20. doi:10.1073/pnas. 1900544116
44. Ayachi M, Niel R, Momken I, Billat V L, Mille-Hamard L. Validation of a Ramp Running Protocol for Determination of the True \dot{V}O₂ₘₐₓ in Mice. Front Physiol. 2016; 7:372. doi: 10.3389/fphys.2016.00372
45. Hakim C H, Wasala N B, Duan D. Evaluation of muscle function of the extensor digitorum longus muscle ex vivo and tibialis anterior muscle in situ in mice. J Vis Exp. 2013; doi: 10.3791/50183
46. Franceschi C, Campisi J. Chronic inflammation (inflammaging) and its potential contribution to age-associated diseases. J Gerontol A Biol Sci Med Sci. 2014; 69 Suppl 1:S4-9. doi: 10.1093/gerona/glu057
47. Rummel C, Sachot C, Poole S, Luheshi G N. Circulating interleukin-6 induces fever through a STAT3-linked activation of COX-2 in the brain. Am J Physiol Regul Integr Comp Physiol. 2006; 291:R1316-26. doi:10.1152/ajpregu.00301.2006
48. Nilsberth C, Elander L, Hamzic N, Norell M, Lonn J, Engstrom L, et al. The role of interleukin-6 in lipopolysaccharide-induced fever by mechanisms independent of prostaglandin E2. Endocrinology. 2009; 150:1850-60. doi: 10.1210/en.2008-0806
49. Ellingsgaard H, Hauselmann I, Schuler B, Habib A M, Baggio L L, Meier D T, et al. Interleukin-6 enhances insulin secretion by increasing glucagon-like peptide-1 secretion from L cells and alpha cells. Nat Med. 2011; 17:1481-9. doi: 10.1038/nm.2513
50. Myers J, Prakash M, Froelicher V, Do D, Partington S, Atwood J E. Exercise capacity and mortality among men referred for exercise testing. N Engl J Med. 2002; 346:793-801. doi: 10.1056/NEJMoa011858
51. Wilson T M, Tanaka H. Meta-analysis of the age-associated decline in maximal aerobic capacity in men: relation to training status. Am J Physiol Heart Circ Physiol. 2000; 278:H829-34. doi:10.1152/ajpheart.2000.278.3.H829
52. Bassett D R, Jr., Howley E T. Limiting factors for maximum oxygen uptake and determinants of endurance performance. Med Sci Sports Exerc. 2000; 32:70-84. doi: 10.1097/00005768-200001000-00012
53. Studenski S, Perera S, Patel K, Rosano C, Faulkner K, Inzitari M, et al. Gait speed and survival in older adults. JAMA. 2011; 305:50-8. doi:10.1001/jama.2010.1923
54. Harris R A, Bowker-Kinley M M, Huang B, Wu P. Regulation of the activity of the pyruvate dehydrogenase complex. Adv Enzyme Regul. 2002; 42:249-59. doi:10.1016/s0065-2571(01)00061-9
55. Linn T C, Pettit F H, Reed L J. Alpha-keto acid dehydrogenase complexes. X. Regulation of the activity of the pyruvate dehydrogenase complex from beef kidney mitochondria by phosphorylation and dephosphorylation. Proc Natl Acad Sci USA. 1969; 62:234-41. doi:10.1073/pnas.62.1.234
56. Yudkin J S, Eringa E, Stehouwer C D. “Vasocrine” signalling from perivascular fat: a mechanism linking insulin resistance to vascular disease. Lancet. 2005; 365:1817-20. doi:10.1016/S0140-6736(05)66585-3
57. Ellingsgaard H, Seelig E, Timper K, Coslovsky M, Soederlund L, Lyngbaek M P, et al. GLP-1 secretion is regulated by IL-6 signalling: a randomised, placebo-controlled study. Diabetologia. 2020; 63:362-73. doi:10.1007/s00125-019-05045-y
58. Wojewoda M, Kmiecik K, Ventura-Clapier R, Fortin D, Onopiuk M, Jakubczyk J, et al. Running performance at high running velocities is impaired but V′O((2)max) and peripheral endothelial function are preserved in IL-6(−)/(−) mice. PLOS One. 2014; 9:e88333. doi:10.1371/journal.pone.0088333
59. Wojewoda M, Kmiecik K, Majerczak J, Ventura-Clapier R, Fortin D, Onopiuk M, et al. Skeletal Muscle Response to Endurance Training in IL-6−/− Mice. Int J Sports Med. 2015; 36:1163-9. doi:10.1055/s-0035-1555851
60. Hawley J A, Lundby C, Cotter J D, Burke L M. Maximizing Cellular Adaptation to Endurance Exercise in Skeletal Muscle. Cell Metab. 2018; 27:962-76. doi:10.1016/j.cmet.2018.04.014
61. San-Millan I, Brooks G A. Assessment of Metabolic Flexibility by Means of Measuring Blood Lactate, Fat, and Carbohydrate Oxidation Responses to Exercise in Professional Endurance Athletes and Less-Fit Individuals. Sports Med. 2018; 48:467-79. doi: 10.1007/s40279-017-0751-x
62. Trenerry M K, Carey K A, Ward A C, Cameron-Smith D. STAT3 signaling is activated in human skeletal muscle following acute resistance exercise. J Appl Physiol (1985). 2007; 102:1483-9. doi: 10.1152/japplphysiol.01147.2006
63. Trenerry M K, Carey K A, Ward A C, Farnfield M M, Cameron-Smith D. Exercise-induced activation of STAT3 signaling is increased with age. Rejuvenation Res. 2008; 11:717-24. doi:10.1089/rej.2007.0643
64. Trenerry M K, Della Gatta P A, Larsen A E, Garnham A P, Cameron-Smith D. Impact of resistance exercise training on interleukin-6 and JAK/STAT in young men. Muscle Nerve. 2011; 43:385-92. doi:10.1002/mus.21875
65. Knudsen N H, Stanya K J, Hyde A L, Chalom M M, Alexander R K, Liou Y H, et al. Interleukin-13 drives metabolic conditioning of muscle to endurance exercise. Science. 2020; 368:doi: 10.1126/science.aat3987
66. Oberhuber M, Pecoraro M, Rusz M, Oberhuber G, Wieselberg M, Haslinger P, et al. STAT3-dependent analysis reveals PDK4 as independent predictor of recurrence in prostate cancer. Mol Syst Biol. 2020; 16:e9247. doi: 10.15252/msb.20199247
67. Mourtzakis M, Saltin B, Graham T, Pilegaard H. Carbohydrate metabolism during prolonged exercise and recovery: interactions between pyruvate dehydrogenase, fatty acids, and amino acids. J Appl Physiol (1985). 2006; 100:1822-30. doi:10.1152/japplphysiol.00571.2005
68. Pilegaard H, Birk J B, Sacchetti M, Mourtzakis M, Hardie D G, Stewart G, et al. PDH-Elalpha dephosphorylation and activation in human skeletal muscle during exercise: effect of intralipid infusion. Diabetes. 2006; 55:3020-7. doi:10.2337/db06-0152
69. Watt M J, Heigenhauser G J, LeBlanc P J, Inglis J G, Spriet L L, Peters S J. Rapid upregulation of pyruvate dehydrogenase kinase activity in human skeletal muscle during prolonged exercise. J Appl Physiol (1985). 2004; 97:1261-7. doi:10.1152/japplphysiol.00132.2004
70. Biensø R S, Knudsen J G, Brandt N, Pedersen P A, Pilegaard H. Effects of IL-6 on pyruvate dehydrogenase regulation in mouse skeletal muscle. Pflugers Arch. 2014; 466:1647-57. doi: 10.1007/s00424-013-1399-5
71. Gudiksen A, Schwartz C L, Bertholdt L, Joensen E, Knudsen J G, Pilegaard H. Lack of Skeletal Muscle IL-6 Affects Pyruvate Dehydrogenase Activity at Rest and during Prolonged Exercise. PLOS One. 2016; 11:e0156460. doi: 10.1371/journal.pone.0156460
72. Faldt J, Wernstedt I, Fitzgerald S M, Wallenius K, Bergström G, Jansson J O. Reduced exercise endurance in interleukin-6-deficient mice. Endocrinology. 2004; 145:2680-6. doi:10.1210/en.2003-1319
73. Lukaszuk B, Bialuk I, Gorski J, Zajaczkiewicz M, Winnicka M M, Chabowski A. A single bout of exercise increases the expression of glucose but not fatty acid transporters in skeletal muscle of IL-6 KO mice. Lipids. 2012; 47:763-72. doi: 10.1007/s11745-012-3678-x
74. Pinto A P, da Rocha A L, Kohama E B, Gaspar R C, Simabuco F M, Frantz F G, et al. Exhaustive acute exercise-induced E R stress is attenuated in IL-6-knockout mice. J Endocrinol. 2019; 240:181-93. doi: 10.1530/JOE-18-0404
75. Chlopicki S, Kurdziel M, Sternak M, Szafarz M, Szymura-Oleksiak J, Kaminski K, et al. Single bout of endurance exercise increases NNMT activity in the liver and MNA concentration in plasma; the role of IL-6. Pharmacol Rep. 2012; 64:369-76.
76. Li L, Mühlfeld C, Niemann B, Pan R, Li R, Hilfiker-Kleiner D, et al. Mitochondrial biogenesis and PGC-1alpha deacetylation by chronic treadmill exercise: differential response in cardiac and skeletal muscle. Basic Res Cardiol. 2011; 106:1221-34. doi: 10.1007/s00395-011-0213-9
77. Christensen R H, Lehrskov L L, Wedell-Neergaard A S, Legaard G E, Ried-Larsen M, Karstoft K, et al. Aerobic Exercise Induces Cardiac Fat Loss and Alters Cardiac Muscle Mass Through an Interleukin-6 Receptor-Dependent Mechanism: Cardiac Analysis of a Double-Blind Randomized Controlled Clinical Trial in Abdominally Obese Humans. Circulation. 2019; 140:1684-6. doi: 10.1161/CIRCULATIONAHA.119.042287
78 Rooks D, Swan T, Goswami B, Filosa L A, Bunte O, Panchaud N, et al. Bimagrumab vs Optimized Standard of Care for Treatment of Sarcopenia in Community-Dwelling Older Adults: A Randomized Clinical Trial. JAMA Netw Open. 2020; 3:e2020836. doi:10.1001/jamanetworkopen.2020.20836
79. Haider S, Grabovac I, Dorner T E. Effects of physical activity interventions in frail and prefrail community-dwelling people on frailty status, muscle strength, physical performance and muscle mass-a narrative review. Wien Klin Wochenschr. 2019; 131:244-54. doi: 10.1007/s00508-019-1484-7
80. Lewsey S C, Weiss K, Schar M, Zhang Y, Bottomley P A, Samuel T J, et al. Exercise intolerance and rapid skeletal muscle energetic decline in human age-associated frailty. JCI Insight. 2020; 5:doi:10.1172/jci.insight. 141246
81. Jadczak A D, Makwana N, Luscombe-Marsh N, Visvanathan R, Schultz T J. Effectiveness of exercise interventions on physical function in community-dwelling frail older people: an umbrella review of systematic reviews. JBI Database System Rev Implement Rep. 2018; 16:752-75. doi: 10.11124/JBISRIR-2017-003551
82. Angulo J, El Assar M, Alvarez-Bustos A, Rodriguez-Manas L. Physical activity and exercise: Strategies to manage frailty. Redox Biol. 2020; 35:101513. doi:10.1016/j.redox.2020.101513
83. Arc-Chagnaud C, Millan F, Salvador-Pascual A, Correas A G, Olaso-Gonzalez G, De la Rosa A, et al. Reversal of age-associated frailty by controlled physical exercise: The pre-clinical and clinical evidences. Sports Med Health Sci. 2019; 1:33-9. doi:10.1016/j.smhs.2019.08.007

What is claimed is:

1. A method of improving muscle performance in a subject with age-related frailty comprising:
a) engaging the subject in an exercise training program; and
b) administering to the subject recombinant IL-6 composition at regular intervals during the exercise training program.

2. The method of claim 1, wherein the method reduces muscle fatigue in the subject.

3. The method of claim 1, wherein the method improves endurance.

4. The method of claim 2, wherein the method improves muscle endurance.

5. The method of claim 1, wherein the method improves muscle fatigue resistance.

6. The method of claim 1, wherein the method improves muscle recovery in the subject.

7. The method of claim 2, wherein the method improves aerobic endurance.

8. The method of claim 1, wherein the method improves maximum aerobic capacity in the subject.

9. The method of claim 1, wherein the method improves motor coordination.

10. The method of claim 1, wherein the method reduces glucose intolerance in the subject.

11. The method of claim 1, wherein the method reduces exercise related lactic acidosis in the subject.

12. The method of claim 1, wherein the method increases the expression of a biomarker selected from the following group of biomarkers in the subject: PDK4, LDHA, ATP5F1A, UQCRC2, MTCO1, and NDUFB5.

13. The method of claim 1, wherein the method decreases white adipose tissue in the subject.

14. The method of any one of claims 1 to 13, wherein the subject is at least about 55 years or older.

15. The method of any one of claims 1 to 14, wherein the exercise training program comprises at least one exercise session a week.

16. The method of claim 15, wherein each exercise session is at least 15 minutes in length.

17. The method of any one of claims 1 to 16, wherein the exercise training program is at least 4 weeks in duration.

18. The method of any one of claims 15 to 17, wherein the subject exerts no more than 65% of the subject's maximum heart rate during each exercise session.

19. The method of any one of claims 15 to 18, wherein recombinant IL-6 is administered to the subject prior to each exercise session.

20. The method of any one of claims 1 to 19, wherein the recombinant IL-6 composition is administered to the subject at regular intervals spaced at least 24 hours apart.

21. The method of any one of claims 1 to 20, wherein the recombinant IL-6 composition is administered at a dose between about 0.05 μg/kg to about 3.0 μg/kg.

22. The method of claim 21, wherein the recombinant IL-6 composition is administered at a dose of between about 0.2 μg/kg to about 1.0 μg/kg.

23. The method of any one of claims 1 to 20, wherein the recombinant IL-6 composition is administered at a dose between about 3.75 μg to about 225.0 μg.

24. The method of claim 23, wherein the recombinant IL-6 composition is administered at a dose of between about 15.0 μg to about 75.0 μg.

25. The method of any one of claims 1 to 24, wherein the IL-6 composition further comprises a pharmaceutically acceptable carrier.

26. The method of any one of claims 1 to 25, wherein the IL-6 composition is administered subcutaneously, intravenously, intramuscularly or orally.

27. The method of any one of claims 1 to 26, wherein the IL-6 composition is co-administered with a supplement comprising vitamin D3, Resveratrol Setmelanotide, VIP, or an iron supplement.
